(19) United States
(12) Patent Application Publication
(10) Pub. No.: US 2017/0198363 A1
(43) Pub. Date: Jul. 13, 2017

(54) COMPOSITIONS AND METHODS FOR DETECTION OF SMALL MOLECULES

(71) Applicants: COLORADO STATE UNIVERSITY RESEARCH FOUNDATION, FORT COLLINS, CO (US); UNIVERSITY OF WASHINGTON, SEATTLE, WA (US)

(72) Inventors: JUNE MEDFORD, WELLINGTON, CO (US); MAURICIO ANTUNES, FORT COLLINS, CO (US); KEVIN MOREY, WINDSOR, CO (US); BENJAMIN JESTER, SEATTLE, WA (US); CHRISTINE TINBERG, SEATTLE, WA (US); STANLEY FIELDS, SEATTLE, WA (US); DAVID BAKER, SEATTLE, WA (US); MATTHEW BICK, SEATTLE, WA (US)

(21) Appl. No.: 15/392,962

(22) Filed: Dec. 28, 2016

Related U.S. Application Data
(60) Provisional application No. 62/271,863, filed on Dec. 28, 2015.

(51) Int. Cl. C12Q 1/68; C12N 9/22; C12N 15/62

(52) U.S. Cl. CPC C12Q 1/6897; C12N 15/625; C12N 9/22

(57) ABSTRACT

Compositions and methods are provided for the detection of small molecules in a cell using biosensor molecules comprising conditionally active ligand binding domains. Compositions for conditionally activating transcription based on the presence of a small molecules in a cell are further provided, together with methods of designing, producing, and expressing biosensor molecules in cells.

### REFERENCE TO RELATED APPLICATIONS

This application claims the benefit of U.S. Provisional Application No. 62/271,863, filed Dec. 28, 2015, which is herein incorporated by reference in its entirety.

### STATEMENT REGARDING FEDERALLY SPONSORED RESEARCH

This invention was made with partial government support by funding from the U.S. Department of Defense/Defense Threat Reduction Agency under grant number numbers W911NF-09-1-0526 and HDTRA1-13-1-0054; funding from the National Institutes of Health under grant number P41 GM103533; funding from the Department of Energy under grant number DE-FG02-02ER63445; funding from the National Science Foundation (NSF)/Synthetic Biology Engineering Research Center (SynBERC) under grant numbers MCB-134189 and EEC-0540879; and funding from the NSF under Graduate Research Fellowship grant number DGE1144152. The Government has certain rights in the invention.

### INCORPORATION OF SEQUENCE LISTING

A sequence listing containing the file named “PHDT003US_ST25.txt” which is 29,678 bytes (measured in MS-Windows®) and created on Dec. 28, 2016, is incorporated herein by reference in its entirety.

### FIELD OF THE INVENTION

The present invention relates to the field of biomedical technology, plant biotechnology and synthetic biology. More specifically, the invention relates to the design and engineering of compositions and methods for the detection of small molecules.

### BACKGROUND OF THE INVENTION

Biosensors capable of sensing and responding to small molecules in vivo have wide-ranging applications in biological research and biotechnology. However, existing strategies for the construction of biosensors have not been sufficiently generalizable to gain widespread use. Existing methods typically couple binding to a single output signal, and use a limited repertoire of natural protein or nucleic acid domains, which narrows the scope of small molecules that can be detected. A need therefore exists for strategies for small molecule detection that are adaptable to a range of small molecules.

### SUMMARY OF THE INVENTION

In one aspect, the invention provides a recombinant polypeptide for the detection of a target ligand in a cell comprising a ligand-binding domain (LBD) capable of binding the target ligand, wherein the recombinant polypeptide has a longer half-life in the presence of the target ligand than in the absence of the target ligand. In certain embodiments, a recombinant polypeptide of the invention further comprises a reporter molecule operably linked to the LBD. The reporter molecule may be a screenable or selectable marker, for example a fluorescent molecule, luciferase, or an enzymatic component. In further embodiments, a recombinant polypeptide of the invention further comprises a DNA-binding domain (DBD) and a transcription activation domain (TAD), each in operable linkage with the LBD.

An LBD of the invention may be a naturally occurring polypeptide, or a variant or fragment thereof with ligand binding activity, or may be a computationally designed to bind the target ligand. 8. For example, an LBD of the invention may be computationally designed to include destabilizing mutations at a homodimer interface of a homodimeric protein or to include mutations that maintain protein structure while altering the specificity of ligand binding.

In certain embodiments, a recombinant polypeptide of the invention comprises an LBD that comprises: a) a polypeptide sequence comprising one or more mutations compared with DIG10.3 (SEQ ID NO:3) and having ligand binding activity; or b) a fragment of DIG10.3 (SEQ ID NO:3) having ligand binding activity. In some examples an LBD of the invention comprises a polypeptide sequence comprising 1, 2, or 3 mutations compared with DIG10.3 (SEQ ID NO:3) and having ligand binding activity. For example, and LBD of the invention may be DIG₀ (SEQ ID NO:5), DIG₁ (SEQ ID NO:7), DIG₂ (SEQ ID NO:9), DIG₃ (SEQ ID NO:11), PRO₀ (SEQ ID NO:13), PRO₁ (SEQ ID NO:15), PRO₂ (SEQ ID NO:17), or PRO₃ (SEQ ID NO:19).

Recombinant polypeptides of the invention may further comprise a degron, for example MATα. Recombinant polypeptides may further recognize a naturally occurring or synthetic upstream activating sequence (UAS) within a promoter.

In some embodiments, a recombinant polypeptide of the invention comprises a DBD that comprises: a) a polypeptide sequence of Gal4 (SEQ ID NO:25) or LexA (SEQ ID NO:29); b) a polypeptide sequence comprising one or more mutations compared with Gal4 Gal4 (SEQ ID NO:25) or LexA (SEQ ID NO:29) and having DNA binding activity; or c) a fragment of Gal4 (SEQ ID NO:25) or LexA (SEQ ID NO:29) having DNA binding activity. In certain examples, a DBD of the invention comprises the sequence of Gal4 (SEQ ID NO:25) or LexA (SEQ ID NO:29).

In further embodiments, a recombinant polypeptide of the invention comprises a TAD that comprises: a) a polypeptide sequence of VP16 (SEQ ID NO:30) or VP64 (SEQ ID NO:31); b) a polypeptide sequence comprising one or more mutations compared with VP16 (SEQ ID NO:30) or VP64 (SEQ ID NO:31) and having transcription activation activity; or c) a fragment of VP16 (SEQ ID NO:30) or VP64 (SEQ ID NO:31) having transcription activation activity. For example, a TAD of the invention may comprise the sequence of VP16 (SEQ ID NO:30) or VP64 (SEQ ID NO:31).

In yet further embodiments, the invention provides recombinant polypeptides wherein the DBD is G_{L77F} (SEQ ID NO:27), the LBD is PRO₁ (SEQ ID NO:15), and the TAD is VP16 (SEQ ID NO:30).

The invention further provides recombinant polypeptides, wherein the LBD comprises: a) a polypeptide sequence of Fen49 (SEQ ID NO:23) or Fen 21 (SEQ ID NO:21); b) a polypeptide sequence comprising one or more mutations compared with Fen49 (SEQ ID NO:23) or Fen 21 (SEQ ID NO:21) and having ligand binding activity; or c) a fragment of Fen49 (SEQ ID NO:23) or Fen 21 (SEQ ID NO:21) having ligand binding activity. For example, LBDs of the invention may comprise a polypeptide sequence of Fen49 (SEQ ID NO:23) or Fen 21 (SEQ ID NO:21). In certain embodiments, the invention provides recombinant polypeptides wherein the DBD is Gal4 (SEQ ID NO:25), the LBD is Fen49 (SEQ ID NO:23), and the TAD is VP16 (SEQ ID NO:30).

In certain embodiments, the invention provides recombinant polypeptides comprising a Cas9 polypeptide sequence operably linked to a LBD.

In a further aspect, the invention provides cells comprising the recombinant polypeptides of the invention, for example mammalian, yeast, or plant cells. The invention further provides polynucleotide sequences encoding the polypeptide sequences of the invention, as well as methods of detecting the presence of a target molecule within a cell comprising introducing recombinant polynucleotides of the invention into the cell and detecting a reporter molecule.

### BRIEF DESCRIPTION OF THE SEQUENCES

SEQ ID NO:1—Gal4-DIG10.3 linker sequence
SEQ ID NO: 2—DIG10.3 nucleotide sequence
SEQ ID NO: 3—DIG10.3 polypeptide sequence
SEQ ID NO: 4—DIG₀ nucleotide sequence
SEQ ID NO: 5—DIG₀ polypeptide sequence
SEQ ID NO: 6—DIG₁ nucleotide sequence
SEQ ID NO: 7—DIG₁ polypeptide sequence
SEQ ID NO: 8—DIG₂ nucleotide sequence
SEQ ID NO: 9—DIG₂ polypeptide sequence
SEQ ID NO: 10—DIG₃ nucleotide sequence
SEQ ID NO: 11—DIG₃ polypeptide sequence
SEQ ID NO: 12—PRO₀ nucleotide sequence
SEQ ID NO: 13—PRO₀ polypeptide sequence
SEQ ID NO: 14—PRO₁ nucleotide sequence
SEQ ID NO: 15—PRO₁ polypeptide sequence
SEQ ID NO: 16—PRO₂ nucleotide sequence
SEQ ID NO: 17—PRO₂ polypeptide sequence
SEQ ID NO: 18—PRO₃ nucleotide sequence
SEQ ID NO: 19—PRO₃ polypeptide sequence
SEQ ID NO: 20—Fen21 nucleotide sequence
SEQ ID NO: 21—Fen21 polypeptide sequence
SEQ ID NO: 22—Fen49 nucleotide sequence
SEQ ID NO: 23—Fen49 polypeptide sequence
SEQ ID NO: 24—GAL4 nucleotide sequence
SEQ ID NO: 25—GAL4 polypeptide sequence
SEQ ID NO: 26—G(L77F) nucleotide sequence
SEQ ID NO: 27—G(L77F) polypeptide sequence
SEQ ID NO: 28—LexA nucleotide sequence
SEQ ID NO: 29—LexA polypeptide sequence
SEQ ID NO: 30-VP16 polypeptide sequence
SEQ ID NO: 31-VP64 polypeptide sequences

### BRIEF DESCRIPTION OF THE DRAWINGS

FIG. 1 shows a schematic of a general method for construction of biosensors for small molecules. (a) Modular biosensor construction from a conditionally destabilized LBD and a genetically fused reporter. The reporter is degraded in the absence but not in the presence of the target small molecule. (b) yEGFP fluorescence of digoxin LBD-GFP biosensors upon addition of 250 μM digoxin or DMSO vehicle. (c) yEGFP fluorescence of progesterone LBD-GFP biosensors upon addition of 50 μM progesterone or DMSO vehicle. (d) Positions of conditionally destabilizing mutations of DIG₀ mapped to the crystal structure of the digoxin LBD (PDB ID 4J9A). Residues are shown as colored spheres and key interactions highlighted in insets. In (b)-(c), fold activation is shown above brackets, (-) indicates cells lacking biosensor constructs, and error bars represent s.e.m. of three technical replicates.

FIG. 2 shows characterization of mutations conferring progesterone-dependent stability. (a) Single-mutant deconvolutions of mutations conferring progesterone sensitivity. The parental biosensor appears in the leftmost column of each panel. (b-d) Positions of mutations in PRO₁ (b), PRO₂ (c), and PRO₃ (d) are mapped to the crystal structure of the digoxin LBD (PDB ID 4J9A) and are shown in colored spheres. (e) Fold activation of PRO₀-GFP biosensors with digoxin biosensor mutations upon addition of 50 μM progesterone.

FIG. 3 shows ligand-dependent transcriptional activation. (a) TF-biosensor construction from a conditionally destabilized LBD, a DNA binding domain and a transactivator domain. (b) Positions of conditionally destabilizing mutations of Gal4 mapped to a computational model of Gal4-DIG₀ homodimer. Residues are shown as colored spheres and key interactions are highlighted in insets. The transactivator domain is not shown. (c) Concentration dependence of response to digoxin for digoxin TF-biosensors driving yEGFP expression. (d) Concentration dependence of response to progesterone for progesterone TF-biosensors driving yEGFP expression. (e) Time dependence of response to 250 μM digoxin for digoxin TF-biosensors. Marker symbols are the same as in c. (f) Time dependence of response to 50 μM progesterone for progesterone TF-biosensors. Marker symbols are the same as in d. In c-f, (-) indicates cells lacking biosensor plasmids and error bars represent s.e.m. of three technical replicates.

FIG. 4 shows improvements to TF-biosensor response. Digoxin-dependent expression of yEGFP by G-DIG₁-V TF-biosensors either (a) containing VP64 or VP16 as the TAD and expressed from a CYC1 promoter or (b) containing a VP16 TAD and expressed from a CYC1, ADH1, or TEF1 promoter. (c) Individual mutations identified in a FACS analysis of an error-prone PCR library of G-DIG-V biosensors were tested for their effect on biosensor function using digoxigenin. Transformants were analyzed in an yEGFP yeast reporter strain containing a deletion in pdr5 (PyE14). Improvements in fold activation relative to parental sequences were localized to mutations in Gal4. (d) R60S and L77F mutations found in Gal4 were introduced into G-DIG₁-V, G-DIG₂-V, and G-PRO₁-V. In each case, the Gal4 mutations had the effect of lowering the amount of luciferase activity in the absence of the relevant ligand.

FIG. 5 shows tuning of TF-biosensors for different contexts. (a) The TAD and DBD of the TF-biosensor and its corresponding binding site in the reporter promoter can be swapped for a different application. Expression of a plasmid-borne luciferase reporter was driven by TF-biosensors containing either a LexA or Gal4 DBD and either a VP16 or B42 TAD. Promoters for the reporter contained DNA-binding sites for either Gal4 or LexA. (b) TF-biosensors were transformed into the yeast strain PJ69-4a and tested for growth on -his minimal media containing 1 mM 3-aminotriazole (3-AT) and the indicated steroid. To determine the effect of including an additional destabilization domain, the degron from Mat-alpha was cloned into one of four positions. (c) G-DIG₁-V biosensor response to digoxigenin in yEGFP reporter strain PyE1 either with or without a deletion to the ORF of PDR5. (d) Ligand and TF-biosensor dependent growth on -his media in yeast strains containing deleted ORFs for efflux related transcription factors (PDR1 and PDR3) or ABC transporter proteins (YOR1, PDR5, SNQ2).

FIG. 6 Application of biosensors to metabolic engineering in yeast. (a) Fold activation of G_{L77F}-PRO₁-V by a panel of steroids in yEGFP reporter strain PyE1. Data are represented as mean±SEM. (b) Growth of degron-G-PRO₁-V in HIS3 reporter strain PJ69-4a is stimulated by progesterone but not pregnenolone. (c) Schematic for directed evolution of 3β-HSD using TF-biosensors for conversion of pregnenolone to progesterone. (d) Fold activation of G_{L77F}-PRO₁-V by a panel of plasmids expressing wild-type 3β-HSD under varying promoter strengths in yEGFP reporter strain PyE1 when incubated in 50 μM pregnenolone. Data for plasmids containing CEN/ARS and 2μ (2 micron) origins are shown. Data are presented as mean±s.e.m. of three technical replicates. (-) indicates cells lacking 3β-HSD. (e) Fold activation of G_{L77F}-PRO₁-V by a panel of evolved 3β-HSD mutants expressed under the TDH3 promoter on a CEN/ARS plasmid and incubated in 50 μM pregnenolone. (f) Progesterone titer in 1 OD of cells produced by strains expressing 3β-HSD mutants. Data are presented as mean±s.e.m. of three biological replicates. Progesterone became toxic at levels of 100 μM and above, leading to substantial cell death. β-estradiol and hydrocortisone were not soluble in yeast growth media at levels above 25 μM. In a and d-f, data are presented as mean±s.e.m. of three biological replicates. In d and e, (-) indicates cells lacking 3β-HSD. *indicates significance with a threshold of p<0.05 using 2-tailed Student's t-test.

FIG. 7 shows how the specificity of PRO biosensors enables selection for auxotrophy complementation. Specificity for progesterone (PRO) over digoxigenin (DIG), digoxin (DGX), digitoxigenin (DTX), pregnenolone (PRE), β-estradiol (B-EST), and hydrocortisone (HYD) for (a) G-PRO₀-V (b) G-PRO₁-V (c) G-PRO₂-V and (d) G-PRO₃-V. (e) Growth response of yeast strain PyE1 transformed with 3β-HSD on CEN/ARS plasmids under various promoters and plated on SC -his (and -ura -leu for plasmid maintenance) containing titrations of 3-AT and either 0.5% DMSO (upper panels) or 50 μM pregnenolone (lower panels). Progesterone became toxic at levels of 100 μM and above, leading to substantial cell death. Beta-estradiol and hydrocortisone were not soluble in yeast growth media at levels above 25 μM. In a-d, error bars represent s.e.m. of three biological replicates.

FIG. 8 shows activation of biosensors in mammalian cells and regulation of CRISPR/Cas9 activity. (a) Concentration dependence of response to digoxin for constructs containing digoxin TF-biosensors and Gal4 UAS-E1b-EGFP reporter individually integrated into K562 cells. G_{R60S,L77F}-PRO₁-V serves as a digoxin insensitive control. (b) Concentration dependence of response to progesterone for constructs containing progesterone TF-biosensors and Gal4 UAS-E1b-EGFP reporter individually integrated into K562 cells. G_{R60S}-DIG₁-V serves as a progesterone insensitive control. (c) Time dependence of response to 100 nM digoxin for constructs containing digoxin TF biosensors and Gal4 UAS-E1b-EGFP reporter individually integrated into K562 cells. G_{R60S,L77F}-PRO₁-V serves as a digoxin insensitive control. (d) Time dependence of response to 25 μM progesterone for constructs containing progesterone TF-biosensors and Gal4 UAS-E1b-EGFP reporter individually integrated into K562 cells. G_{R60S}-DIG₁-V serves as a progesterone insensitive control. (e) DIG₃ and PRO₁ fused to the N-terminus of S. pyogenes Cas9 were integrated into a K562 cell line containing a broken EGFP. EGFP function is restored upon transfection of a guide RNA and donor oligonucleotide with matching sequence in the presence of active Cas9. Data are presented as mean±s.e.m. across three biological replicates.

FIG. 9 shows application of biosensors in plants. (a) Activation of luciferase expression in transgenic Arabidopsis plants containing the G-DIG₁-V biosensor in the absence (left) or presence (right) of 100 μM digoxin. Luciferase expression levels are false colored according to scale to the right. (b) Brightfield image of plants shown in (a).

FIG. 10 shows the characterization of DIG biosensor in plants. (a) Test of DIG₀ variants engineered for plant function in Arabidopsis protoplasts. Two activation domains TADs, VP16 (V) and VP64 (VP64), as well as two degrons, yeast MATα and Arabidopsis DREB2a, were added to D_{TF}-1 (G-DIG₀), and the proteins were constitutively expressed from the CaMV35S promoter. The Gal4-activated pUAS promoter controls expression of a luciferase reporter. Transformed protoplasts were incubated with digoxigenin at 0, 100 μM, and 500 μM for 16 hours. (b) Digoxigenin-dependent activation of luciferase expression in three independent transgenic Arabidopsis lines. Plants were incubated in the absence (Control) or presence (DIG) of 100 μM digoxigenin for 42 hours and imaged. Quantification of luciferase expression is presented as mean relative luciferase units±s.d. of ten plants. (c) Digoxigenin dose response curve in transgenic Arabidopsis plants. Concentrations are expressed in micromolar. Data are presented as mean fold induction relative to the control±s.e.m. of ten technical replicates. (d) Specificity of luciferase activation in transgenic Arabidopsis plants. All inducers were tested at 100 μM concentration. DIG, digoxigenin; DIGT, digitoxigenin; β-EST, β-Estradiol. Data are presented as mean fold activation relative to the control±s.e.m. of ten technical replicates.

FIG. 11 shows a schematic of biosensor platform. (a) Biosensors for small molecules are modularly constructed by replacing the LBD with proteins possessing altered substrate preferences. (b) Activity of the biosensor can be tuned by 1) introducing destabilizing mutations (red Xs), 2) adding a degron, 3) altering the strength of the TAD or DNA binding affinity of the TF, 4) changes in the number of TF binding sites or sequences, and 5) titrating 3-aminotriazole, an inhibitor of His3. (c) Yeast provide a genetically tractable chassis for biosensor development prior to implementation in more complex eukaryotes, such as mammalian cells and plants.

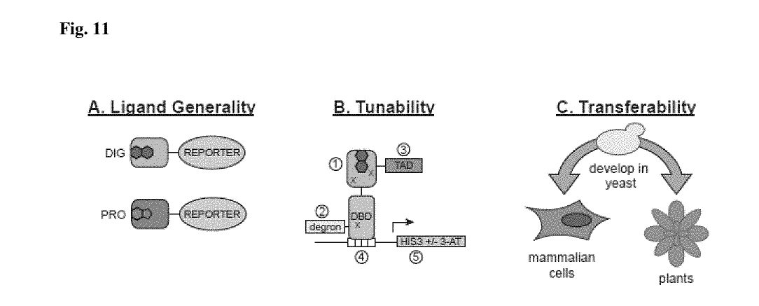

FIG. 12 shows fentanyl-dependent transcriptional activation in Arabidopsis thaliana. (A, B) Protoplasts expressing conditionally stable transcription-factors (TF) Fen21.3 (A) and Fen49.3 (B) driving expression of firefly luciferase respond to treatment with fentanyl. Control cells did not receive fentanyl. Fen21 (˜8-fold luciferase expression over background) was found to be more responsive to fentanyl compared with Fen49, and was used to generate stable transgenic plants. (C) Heterozygous transgenic plants (T₁ generation) stably expressing the Fen21 TF showed increased firefly luciferase expression in the presence of 500 μM fentanyl over 48 hours of exposure. (D) Images of luciferase expression in transgenic plants expressing Fen21 TF in the absence (Control) and presence (Fentanyl) of 500 μM fentanyl. Pixel intensity in luciferase images (bottom row) is false colored according to scale to the right.

FIG. 13 shows function of progesterone biosensor in plant cells (protoplasts). Four progesterone binding sensor proteins (PRO₁-PRO₄) were tested for activation of luciferase expression in plant protoplasts. As observed in yeast cells, PRO₂ had the lowest background activity, and showed the highest increase in luciferase expression in the presence of 25 μM progesterone. This activity was enhanced by the L77F mutation in the Gal4 DBD, resulting in an ˜3-fold increase over background levels. Progesterone concentration of 250 μM seems to be toxic to the plant cells.

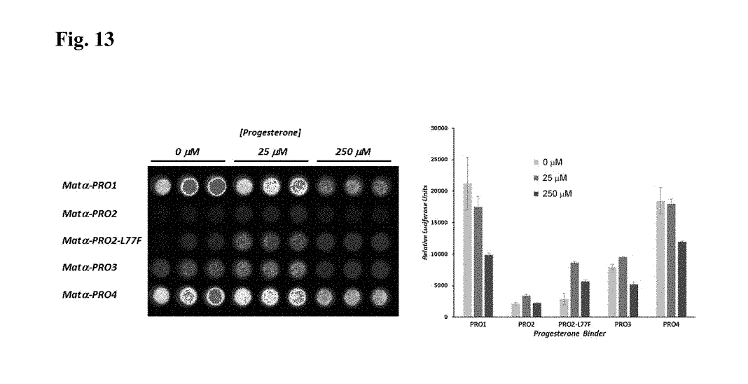

### DETAILED DESCRIPTION

The ability to detect and respond to the presence of small molecules in a cell has wide-ranging applications in biological research and biotechnology, including metabolic pathway regulation, biosynthetic pathway optimization, metabolite concentration measurement and imaging, environmental toxin detection, small molecule-triggered therapeutic response, plant sentinels, plants that sense a molecule and respond, and plants that sense molecules and activate production of a response such as those listed above and/or an environmental remediation response. Despite the need for such methods, previous strategies have relied on a limited repertoire of naturally occurring proteins or nucleic acid binding domains, which narrows the scope of small molecules that can be detected.

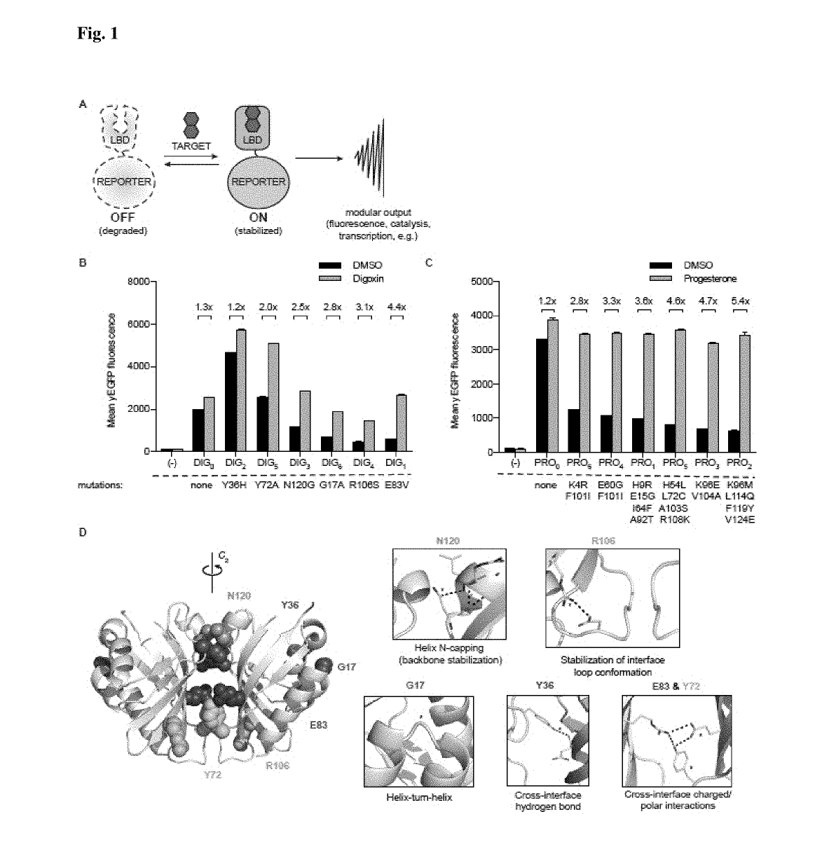

In order to overcome these limitations in the field, the present invention provides a general approach to biosensor design using conditionally stable ligand-binding domains (LBDs). In the absence of a cognate ligand, these proteins are degraded by the ubiquitin proteasome system. Binding to the ligand stabilizes the LBD and prevents degradation. Fusing the destabilized LBD to a suitable reporter protein, such as an enzyme, fluorescent protein, or transcription factor, gene editing systems (e.g., CRISPR/Cas9) renders the fusion conditionally stable and generates sensor response (FIG. 1a). The invention provides LBDs derived from naturally occurring proteins that are engineered to be conditionally stable in the presence of a target ligand or LBDs that are computationally designed for small molecules. Thus, the present invention provides methods for designing an LBD to be used in cases for which natural binding proteins do not exist or lack sufficient specificity or bio-orthogonality.

The invention therefore provides biosensor polypeptide molecules comprising conditionally stable LBDs capable of detecting the presence of a target small molecule in a cell or intact plant. In general, the biosensors provided herein comprise a conditionally stable LBD operably linked to a reporter molecule or a transcription activation molecule allowing for detection of a bound ligand in the LBD. In certain embodiments, the invention provides biosensor molecules comprising a LBD operably linked with a reporter molecule such as a fluorescent molecule. In further embodiments, the invention provides biosensor molecules comprising a conditionally stable LBD operably linked to a DNA binding domain (DBD) and a transcription activation domain (TAD), allowing for activation of transcription of a detectable linked coding sequence when the LBD is stabilized by the presence of the target molecule.

The activity of the biosensor molecules of the invention can be altered to modulate the activity and specificity of the biosensor, for example by: 1) introducing destabilizing or stabilizing mutations to the LBD; 2) adding a degron within the biosensor molecule; 3) altering the strength of the TAD or DNA binding affinity of the DBD, 4) altering the number of DBD binding sites or sequences in a recombinant promoter region recognized by the DBD; 5) altering the specificity through computationally design of the LBD

In certain examples described herein, conditionally stable LBDs of the invention are used to engineer highly specific biosensors for the clinically relevant steroids digoxin and progesterone, or used in genetic circuits for detection responses in intact plants. The invention further provides LBDs fused to fluorescent reporters to be conditionally stable in a cell. Biosensors comprising LBDs operably linked to DBDs and TADs and capable of increasing activating transcription in the presence of a target small molecule are further provided. In some embodiments, biosensor molecules of the invention are capable of detecting the presence of a target small molecule at between 1 nM and 1 mM concentrations, for example between 1 nM and 1 μM, or between 1 nM and 100 nM, or between 1 nM and 10 nM. In other embodiments, biosensor of the present invention are capable of increasing transcription of a coding sequence up to 100-fold in the presence of a target ligand relative to levels in the absence of the target ligand. Biosensors of the present invention may be optimized for use in any cell type, such as for mammalian and plant cells as shown herein. In further embodiments, the invention provides methods of detecting small molecules in a cell and methods of modulating transcription of specific sequences using the biosensor molecules provided herein.

I. Biosensors

In certain embodiments, the present invention provides chimeric polypeptides having biosensor activity. Biosensor polypeptides of the invention comprise a ligand-binding domain (LBD) which may be operably linked to a reporter molecule or transcriptional activator. LBDs for use in biosensors of the invention may be naturally occurring LBDs, computationally designed LBDs, or variant LBDs destabilized by mutation, such that the chimeric biosensor polypeptide accumulates only in cells containing a target ligand due to stabilization of the LBD by ligand binding. Conditionally-destabilized LBDs, biosensors comprising the LBDs, and methods for designing conditionally-destabilized LBDs and biosensors are provided by the invention.

As used herein, “ligand-binding domain,” or “LBD,” refers to a polypeptide capable of binding to a ligand or target molecule. The LBD can be computationally designed. Binding may be covalent or non-covalent, and may occur via the interaction of one or more surfaces of the LBD with the target molecule. In certain embodiments of the invention, chimeric polypeptide biosensors comprise LBDs which render the biosensor conditionally stable in a cell. For example, LBDs for use in the present invention may be destabilized, such as by the introduction of mutations, such that the fusion accumulates only in cells containing the target ligand and is degraded in the absence of the target ligand. In certain embodiments, mutations that stabilize a LBD are in the dimer interface of a homodimeric protein such that the mutations destabilize the homodimer interface to produce a destabilized LBD.

As used herein, a “target” or “target ligand” or “target molecule” refers to a molecule capable of binding a LBD or a conditionally stable LBD of the invention. The conditional stability of a LBD within a chimeric biosensor polypeptide of the invention allows for activation of a reporter molecule or transcriptional activator when a target ligand is present.

A LBD for use in a biosensor polypeptide of the invention may be any molecule capable of binding a target molecule. LBDs for use in biosensors of the invention may be designed using naturally occurring molecules or computationally designed scaffolds, for example by introducing mutations into a molecule to create a conditionally stable LBD. In certain embodiments of the invention, LBDs for use in biosensors are designed based on the computationally designed DIG10.3 scaffold (SEQ ID NO:3), and may include one or more mutations relative to the DIG10.3 sequence that increase or decrease the affinity of the LBD digoxin or other target ligands relative to the DIG10.3 sequence. The invention further provides LBDs for use in biosensors designed based on the modified DIG10.3 sequence PRO₀ (SEQ ID NO:13) that may include one or more mutations relative to the PRO₀ sequence that increase or decrease the affinity of the LBD progesterone or other target ligands relative to the PRO₀ sequence. Examples of LBDs for use in biosensors of the invention include DIG10.3, DIG⁰ (SEQ ID NO:5), DIG₁ (SEQ ID NO:7), DIG₂ (SEQ ID NO:9), DIG₃ (SEQ ID NO:11), PRO₀ (SEQ ID NO:13), PRO₁ (SEQ ID NO:15), PRO₂ (SEQ ID NO:17), or PRO₃ (SEQ ID NO:19), with or without mutations to the sequences to optimize binding affinity for the target ligand. LBDs for use in biosensors of the invention further comprise variants or fragments of DIG10.3, DIG₀ (SEQ ID NO:3), DIG₁ (SEQ ID NO:4), DIG₂ (SEQ ID NO:5), DIG₃ (SEQ ID NO:6), PRO₀ (SEQ ID NO:7), PRO₁ (SEQ ID NO:8), PRO₂ (SEQ ID NO:9), or PRO₃ (SEQ ID NO:10), such as variants comprising 1, 2, 3, 4, 5, 6, 7, 8, 9, or 10 mutations relative to the base sequence, wherein the variants have target ligand binding activity.

In further embodiments, LBDs are computationally designed by surveying large numbers of protein scaffolds, for example from a publically available database such as RCSB Protein Data Bank, an information portal to 3D shape of biological macromolecular structures or Binding MOAD (Hu, Proteins 60:3, pp. 333-340, 2005), based on calculated affinity for a target ligand structure. In specific embodiments, conditionally stable LBDs are selected from a database of molecular scaffolds based on affinity for the structure of fentanyl-citrate toluene solvate or conformers thereof. Examples of LBDs for use in biosensors of the invention include Fen21 (SEQ ID NO:21) and Fen49 (SEQ ID NO:23). Methods of computationally designing polypeptides including LBDs with certain affinities or specificities for a target ligand are known in the art and described, for example, in Bale et al. Science 353, 389-394, 2016; Bhardwaj et al. Nature 538, 329-335, 2016; Boyken et al. Science, 352, 680-687, 2016; Horowitz et al. Nat Comm 7, 12549, 2016; Hsia et al. Nature, 535, 136-139, 2016; Huang et al. Nature 537, 320-327, 2016; Huang et al. Nat Chem Biol, 12, 29-34, 2016; Mills et al. Proc Natl Acad Sci USA 2016, Rose et al. Nat Chem Biol 13, 119-126 2017; Taylor et al. Nat Methods, 13, 177-183, 2016.

In certain embodiments, LBDs are altered to be conditionally stable in the presence of a target ligand. A naturally occurring or designed LBD may be further engineered by the introduction of random or rationally designed mutations into the LBD. Mutations may be within the binding site of a LBD, or within other regions of the LBD, for example within regions associated with dimerization. Mutations or alterations to a LBD may be introduced using any method known in the art, for example by the use of random mutagenesis or error-prone PCR to alter a polynucleotide sequence encoding the LBD. Candidate LBDs may be tested for binding affinity with a target ligand by methods known the art, including binding assays using fluorescent or other reporter molecules. Additional approaches for designing binding proteins with high affinity and selectivity include designing pre-organized and shape complementary to small molecule binding sites are provided, for example, in Tinberg et al. Nature, 501, 212-216, 2013.

In further embodiments, biosensor polypeptides of the invention comprise a DNA-binding domain (DBD) operably linked to a conditionally stable LBD and operably linked to a transcription activation domain (TAD). In certain embodiments, chimeric biosensors comprise an N-terminal DBD operably linked with an LBD that is operably linked with a C-terminal TAD.

As used herein, a “DNA-binding domain” or “DBD” refers to a molecule, such as a polypeptide sequence, that is capable of binding to a polynucleotide sequence. DBDs typically recognize one or more consensus sequences within a DNA strand, for example a synthetic or naturally occurring upstream activating sequence (UAS) within a promoter. A DBD for use in a biosensor of the invention may bind DNA with high or low affinity. Examples of DBDs for use in the invention include Gal4 (SEQ ID NO:25) or LexA (SEQ ID NO:29) DBDs, or variants or fragments of Gal4 or LexA DBDs comprising altered DNA-binding activity. DBDs that are also relevant include any naturally occurring DBDs or various synthetic DBDs such as zinc fingers, TAL Effectors, CRISPR/Cas9 or variants thereof, as well as computationally designed DBDs including designed Cas9 sequences with corresponding guide RNAs.

As used herein, a “transcription activation domain” or “TAD” refers to a molecule, such as a polypeptide sequence, that is capable of activating transcription of a polynucleotide sequence. In certain examples, TADs interact with a promoter region associated with a coding sequence to initiate transcription of the coding sequence by RNA polymerase. TADs typically comprise conserved residues, for example acidic or hydrophobic residues, involved in promoting the transcription of a coding sequence. Examples of TADs for use in the present invention include VP16 (SEQ ID NO:30) or VP64 (SEQ ID NO:31) TADs, or variants or fragments VP16 or VP64 TADs comprising altered transcription activation activity. TADs that are also relevant include any naturally occurring TADs, TADs such as those from Tal Effectors, or synthetic TADs such as computationally designed TADs.

In further embodiments, biosensor polypeptides of the invention comprise a degron capable of modulating the stability of the biosensor polypeptide. Examples of degrons include Matα and DREB2a as well as those involved in highly regulated proteins such as proteins involved in cell division, cell replication, light sensing, and hormonal responses. Further examples are sequences for site specific ubiquitination or other secondary modification that targets a protein for degradation.

Biosensors of the invention comprising LBDs as described herein thus provide for conditional activation of a reporter molecule, or conditional transcription and expression of a polynucleotide sequence, depending on the presence or absence of a target ligand. The invention further provides methods of designing biosensors with improved sensitivity to the presence of a target small molecule or which are capable of amplifying ligand-dependent activation of transcription, as described herein.

II. Recombinant Biosensor Molecules

The present invention provides recombinant biosensor molecules, such as recombinant polypeptides, comprising conditionally stable LBDs. As used herein, the term “recombinant” refers to a non-natural polynucleotide, polypeptide, or organism that would not normally be found in nature and was created by human intervention. As used herein, a “recombinant polypeptide molecule” is a polypeptide molecule comprising a combination of polypeptide molecules that would not naturally occur together and is the result of human intervention, for example, a polypeptide molecule that is comprised of a combination of at least two polypeptide molecules heterologous to each other. An example of a recombinant polypeptide molecule is a biosensor polypeptide molecule as described herein comprising a LBD of the invention operably linked to a heterologous reporter molecule, DBD, or TAD. An example of a recombinant polynucleotide molecule is a polynucleotide molecule encoding a biosensor polypeptide molecule as described herein. As used herein, a “recombinant plant” is a plant that would not normally exist in nature, is the result of human intervention, and contains a recombinant polynucleotide or polypeptide, for example through the integration of a heterologous polynucleotide into the genome of the plant. As a result of such genomic alteration, the recombinant plant is something new and distinctly different from the related wild-type plant.

As used herein, the term “heterologous” refers to a first molecule not normally associated with a second molecule or an organism in nature. For example, a first polynucleotide molecule from a first source may be operably linked to a second polynucleotide molecule from a second source directly or via a linker molecule. In another example, a first polynucleotide molecule may be derived from a first species and inserted into the genome of a second species. The polynucleotide molecule would then be heterologous to the genome and the organism.

As used herein, the term “chimeric” refers to a single polypeptide molecule produced by fusing a first polypeptide molecule to a second polypeptide molecule, where the polypeptide molecules would not normally be found in that configuration fused to one another. The chimeric polypeptide molecule is thus a new polypeptide molecule not normally found in nature. Similarly, a chimeric polynucleotide molecule may be produced by fusing a first polynucleotide from a first source with a second polynucleotide from a second source to form a single polynucleotide molecule. The biosensor polypeptide molecules of the present invention are examples of chimeric polypeptides.

As used herein, the term “isolated polynucleotide molecule” or “isolated polypeptide molecule” refers to a DNA molecule at least partially separated from other molecules normally associated with it in its native or natural state. In one embodiment, the term “isolated” refers to a polynucleotide molecule that is at least partially separated from some of the nucleic acids which normally flank the DNA molecule in its native or natural state. Thus, polynucleotide molecules fused to regulatory or coding sequences with which they are not normally associated, for example as the result of recombinant techniques, are considered to be “isolated.” Such molecules are considered isolated when integrated into the chromosome of a host cell or present in a nucleic acid solution with other polynucleotide molecules, in that they are not in their native state. Similarly, polypeptide molecules fused to heterologous polypeptide molecules to form a recombinant polypeptide molecule are considered to be “isolated.”

Any number of methods well known to those skilled in the art can be used to isolate and manipulate a DNA molecule, or fragment thereof, as disclosed in the present invention. For example, polymerase chain reaction (PCR) technology can be used to amplify a particular starting DNA molecule and/or to produce variants of the original molecule. DNA molecules, or fragment thereof, can also be obtained by other techniques, such as by directly synthesizing the fragment by chemical means, as is commonly practiced by using an automated oligonucleotide synthesizer. Similarly, methods well known in the art can be used to isolate or manipulate polypeptide molecules, including the production of recombinant polynucleotide molecules encoding a desired polypeptide molecule.

As used herein, the term “sequence identity” refers to the extent to which two optimally aligned polynucleotide sequences or two optimally aligned polypeptide sequences are identical. An optimal sequence alignment is created by manually aligning two sequences, e.g. a reference sequence and another sequence, to maximize the number of nucleotide matches in the sequence alignment with appropriate internal nucleotide insertions, deletions, or gaps. As used herein, the term “reference sequence” refers to a sequence provided as the polynucleotide sequences of SEQ ID NOs: 3, 5, 7, 9, 11, 13, 15, 17, 19, 21, 23, 25, 29, 30, or 31.

As used herein, the term “percent sequence identity” or “percent identity” or “% identity” is the identity fraction multiplied by 100. The “identity fraction” for a sequence optimally aligned with a reference sequence is the number of nucleotide or amino acid matches in the optimal alignment, divided by the total number of nucleotides or amino acids in the reference sequence, e.g. the total number of nucleotides or amino acids in the full length of the entire reference sequence. Thus, in one embodiment, the invention provides a polynucleotide molecule comprising a sequence that, when optimally aligned to a reference sequence, provided herein as 3, 5, 7, 9, 11, 13, 15, 17, 19, 21, 23, 25, 29, 30, or 31, has at least about 85 percent identity, at least about 90 percent identity, at least about 95 percent identity, at least about 96 percent identity, at least about 97 percent identity, at least about 98 percent identity, or at least about 99 percent identity to the reference sequence. The invention further provides a polypeptide molecule comprising a sequence that, when optimally aligned to a reference sequence, provided herein as SEQ ID NOs: 3, 5, 7, 9, 11, 13, 15, 17, 19, 21, 23, 25, 29, 30, or 31, has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments, such sequences may be defined as having ligand binding activity.

In one embodiment, fragments are provided of a reference polypeptide sequence disclosed herein. Polypeptide fragments may comprise the activity of a reference sequence and may be useful alone or in combination with other recombinant polypeptides of the invention, such as in constructing chimeric biosensor polypeptides. In specific embodiments, fragments of a reference sequence are provided comprising at least about 5, 10, 15, 20, 25, 30, 40 50, 95, 150, 250, or at least about 500 contiguous amino acid residues, or longer, of a reference polypeptide molecule and having ligand binding activity, transcription activation activity, or DNA binding activity of the reference sequence.

Recombinant polynucleotide or polynucleotide molecules of the invention, including recombinant LBD polypeptides, may further comprise mutations relative to a reference sequence. A recombinant polynucleotide or polypeptide comprises “mutations” if it includes one or more altered nucleotides or amino acids relative to a reference sequence. According to embodiments of the invention, the presence of mutations relative to a polypeptide reference sequence may increase, decrease, or maintain the ligand binding activity of a polypeptide relative to a reference sequence. In certain embodiments of the invention, a polynucleotide or polypeptide sequence may comprise 1, 2, 3, 4, 5, 6, 7, 8, 9, 10, or more mutations relative to a reference sequence. Polypeptides comprising mutations may exhibit increased, decreased, or maintained ligand binding activity.

II. Biosensor Constructs

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule, where one or more polynucleotide molecule has been linked in a functionally operative manner, i.e. operably linked. As used herein, the term “vector” means any recombinant polynucleotide construct that may be used for the purpose of transformation, i.e. the introduction of heterologous DNA into a host cell. A vector according to the present invention may include an expression cassette or transgene cassette isolated from any of the aforementioned molecules. Expression cassettes or transgene cassettes useful in practicing the invention comprise sequences encoding biosensor polypeptides as described herein, for example comprising LBDs, DBDs, TADs, or degrons described herein.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell.

Methods are known in the art for assembling and introducing constructs into a cell in such a manner that a transcribable polynucleotide molecule is transcribed into a functional mRNA molecule that is translated and expressed as a protein product. For the practice of the present invention, conventional compositions and methods for preparing and using constructs and host cells are well known to one skilled in the art (see, for example, Molecular Cloning: A Laboratory Manual, 3^{rd} edition Volumes 1, 2, and 3, J. Sambrook, D. W. Russell, and N. Irwin, Cold Spring Harbor Laboratory Press, 2000). Methods for making recombinant vectors particularly suited to plant transformation include, without limitation, those described in U.S. Pat. Nos. 4,971,908; 4,940,835; 4,769,061; and 4,757,011 in their entirety. These types of vectors have also been reviewed in the scientific literature (see, for example, Rodriguez, et al., Vectors: A Survey of Molecular Cloning Vectors and Their Uses, Butterworths, Boston, 1988; and Glick et al., Methods in Plant Molecular Biology and Biotechnology, CRC Press, Boca Raton, Fla., 1993). Typical vectors useful for expression of nucleic acids in higher plants are well known in the art and include vectors derived from the tumor-inducing (Ti) plasmid of A. tumefaciens (Rogers et al., Methods in Enzymology 153: 253-277, 1987). Other recombinant vectors useful for plant transformation, including the pCaMVCN transfer control vector, have also been described in the scientific literature (see, for example, Fromm et al., Proc. Natl. Acad. Sci. USA 82: 5824-5828, 1985).

Various regulatory elements may be included in a construct including any of those provided herein. Any such regulatory elements may be provided in combination with other regulatory elements. Such combinations can be designed or modified to produce desirable regulatory features. In one embodiment, constructs of the present invention comprise at least one regulatory element operably linked to a transcribable polynucleotide molecule operably linked to a 3′ transcription termination molecule.

Constructs provided by the invention may further comprise a report molecule, such as a screenable or selectable marker molecule. Screenable or selectable markers are known in the art. Commonly used selectable marker genes include those conferring resistance to antibiotics such as kanamycin and paromomycin (nptII), hygromycin B (aph IV), spectinomycin (aadA) and gentamycin (aac3 and aacC4). Markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Biosensor constructs or biosensor polynucleotides of the present invention may be introduced into a host cell using any method known in the art for introducing a polynucleotide or polypeptide into a cell. In certain examples, biosensor constructs of the invention may be introduced into a host cell via transformation, and such constructs may be transiently expressed or stably integrated into the genome of the cell. Biosensor polypeptides may be introduced into a host cell through any method known in the art, or may be expressed within a host cell or assembled within a host cell. Host cells suitable for use in the present invention include, but are not limited to any bacterial, yeast, animal or plant cell, for example mammalian cells or any species of plant cell.

III. Biosensor Applications

The present invention further provides methods of using the biosensor polypeptide molecules provided by the invention. In certain examples, the invention provides methods of detecting small molecules in a cell, modulating transcription, or regulating genome editing using biosensor polypeptides.

In some embodiments, the invention provides methods of detecting small molecules using a biosensor polypeptide, for example a biosensor polypeptide comprising a conditionally stable LBD as described herein operably linked with a reporter molecule. In certain embodiments, reporter molecules of the present invention may be fluorescent molecules, luciferase, or an enzymatic component, for example an enzymatic component involved in the ratio of chlorophyll A to chlorophyll B, enzymatic components such as those involved in production of pigments, or enzymatic components such as those involved in the biological production of heat. In specific examples, biosensor described herein can be used to detect and measure the concentration of small molecules within a cell. This includes applications such as metabolite concentration measurement and imaging, environmental toxin detection, and small molecule-triggered therapeutic response.

In further embodiments, the invention provides methods for producing a small molecule-triggered therapeutic response in a cell. In specific examples, biosensors of the present invention may enable a cell to respond to the presence of a small molecule, such as a pollutant or toxin by activating transcription of an appropriate transcribable polynucleotide sequence, or leading to other types of biological responses including stability of biological molecules. Coupling biosensors with a phytoremediation trait could enable plants to both sense a contaminant and activate a bioremediation gene circuit. When paired with an agronomic or biofuel trait, such biosensors could serve as triggers for bioproduction.

In yet further embodiments, the invention provides methods for conditional activation of a genome editing system in a cell. In certain embodiments provided herein, chimeric Cas9 biosensor polypeptides are conditionally activated in the presence of a target ligand of a LBD within the biosensor polypeptide.

### EXAMPLES

### Example 1

### Design of Fluorescent Biosensors Using Engineered Ligand-Binding Domains

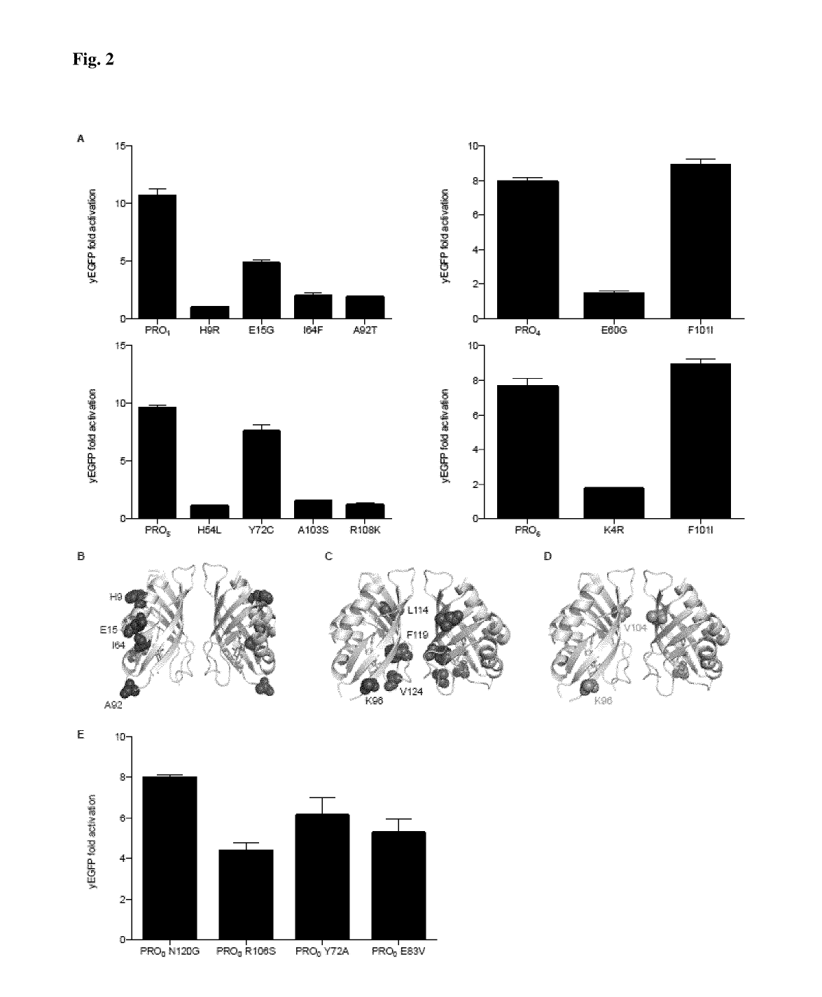

Ligand-binding domains (LBDs) intended for biosensor development should recognize their targets with high affinity and specificity. The computationally designed binding domain DIG10.3 (Tinberg, et al., Nature 501, 212-6, 2013), hereafter DIG₀, was used, which binds the plant steroid glycoside digoxin and its aglycone digoxigenin with picomolar affinities. Introduction of three rationally designed binding site mutations into DIG₀ resulted in a progesterone binder (PRO₀) with nanomolar affinity. Genetic fusions of DIG₀ and PRO₀ to a yeast-enhanced GFP (LBD-biosensors DIG₀-GFP and PRO₀-GFP) were constructed and constitutively expressed in S. cerevisiae. The fusions showed little change in fluorescence in response to digoxin or progesterone, respectively (FIG. 1b). The LBDs of DIG₀-GFP and PRO₀-GFP were randomly mutagenized by error-prone PCR and subjected libraries of 105 integrants to multiple rounds of FACS, sorting alternately for high fluorescence in the presence of the ligand and low fluorescence in its absence. LBD variants having greater than 5-fold activation by cognate ligand (FIGS. 1b and 1c) were isolated. By making additional variants that contain single mutations of the up to four mutations found in the progesterone biosensors, it was shown that some mutations are additive, while others predominately contribute to sensitivity (FIGS. 1 and 2a).

Many of the conditionally-destabilizing mutations identified for DIG₀ involve residues participating in key dimer interface interactions (FIG. 1d). The conditionally-destabilizing mutations of PRO₀ are located throughout the protein (FIGS. 1 and 2b-d); the DIG₀ interface mutations also rendered PRO₀-GFP conditionally stable on binding progesterone (FIGS. 1 and 2e).

### TF-Biosensors Amplify Ligand-Dependent Responses

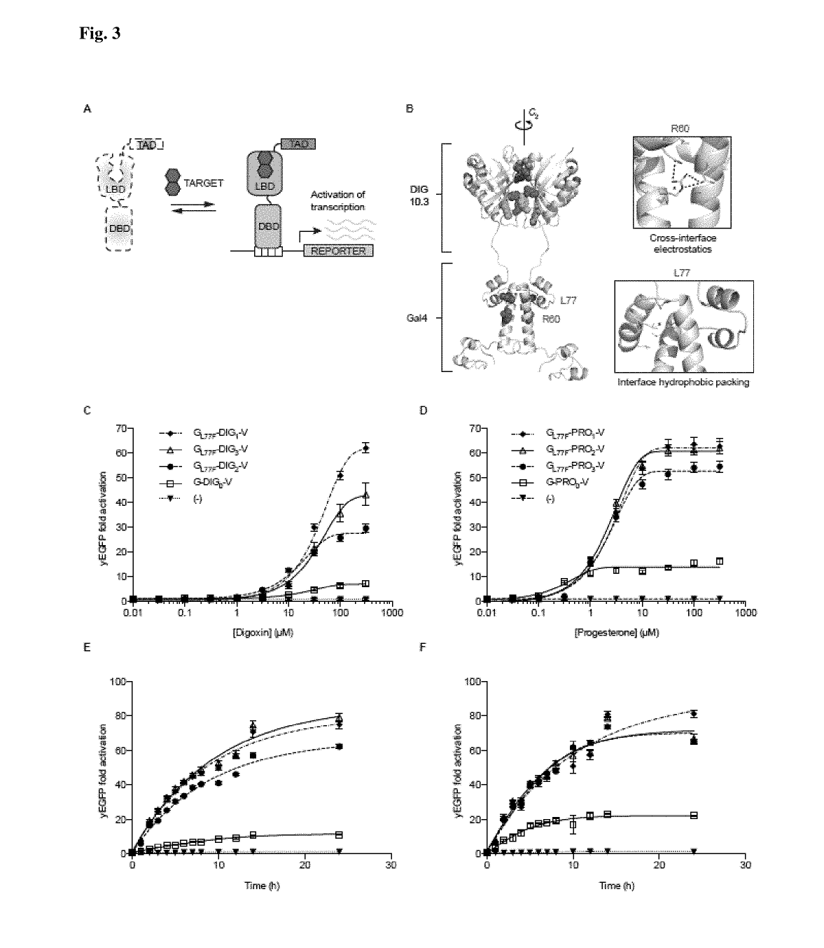

To improve the dynamic range and utility of the biosensors, conditionally stable LBD transcription factor fusions (TF-biosensors) were built by placing an LBD between an N-terminal DNA-binding domain (DBD) and a C-terminal transcriptional activation domain (TAD, FIG. 3a).

The use of TFs serves to amplify biosensor response and allows for ligand-dependent control of gene expression. The initial constructs used the DBD of Gal4, the destabilized LBD mutant DIG₁ (E83V), and either the TAD VP16 or VP64 to drive the expression of yEGFP from a GAL1 promoter. The dynamic range of TF-biosensor activity was maximal when the biosensor was expressed using a weak promoter and weak activation domain because of lower background activity in the absence of ligand (FIGS. 2a, 2b, and 3).

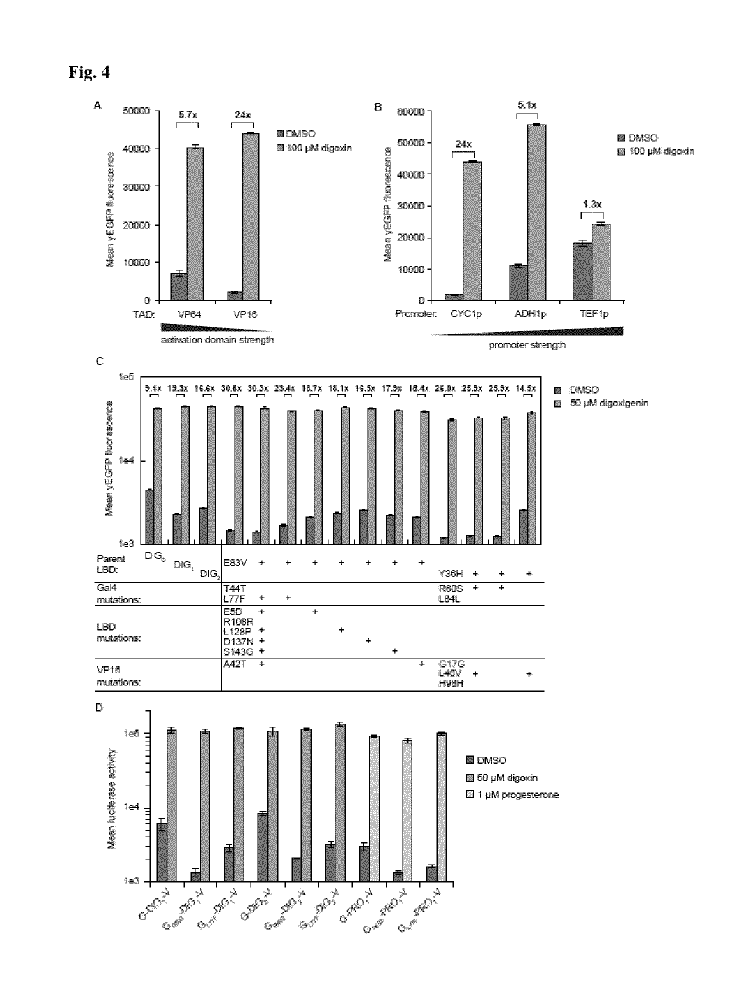

Gal4-DIG₁-VP16 (hereafter G-DIG₁-V) was chosen for further TF-biosensor development because it has both a large dynamic range and maximal activation by ligand. A FACS-based screen of an error-prone PCR library of G-DIG₀-V, G-DIG₁-V, and G-DIG₂-V variants identified mutations L77F and R60S in the Gal4 dimer interface (hereafter GL77F, GR60S) that further increased TF biosensor response by lowering background activity in the absence of ligand (FIG. 3b and FIG. 4c). Although these Gal4 mutations were identified by screening libraries of digoxin-dependent TF-biosensors, they also increased progesterone-dependent activation of the G-PRO-V series of biosensors, indicating a shared mechanism of conditional stability in both systems (FIG. 4d). Combining mutations in Gal4 and DIG or PRO led to activations of up to 60-fold by cognate ligand, a ten-fold improvement over the most responsive LBD-biosensors (FIG. 3c,d), and a dynamic range that has been challenging to achieve in yeast. The TF-biosensors were also rapidly activated, showing a fivefold increase in signal after one hour of incubation with ligand and full activation after ˜14 hours (FIG. 3e,f).

### TF-Biosensors are Tunable and Modular

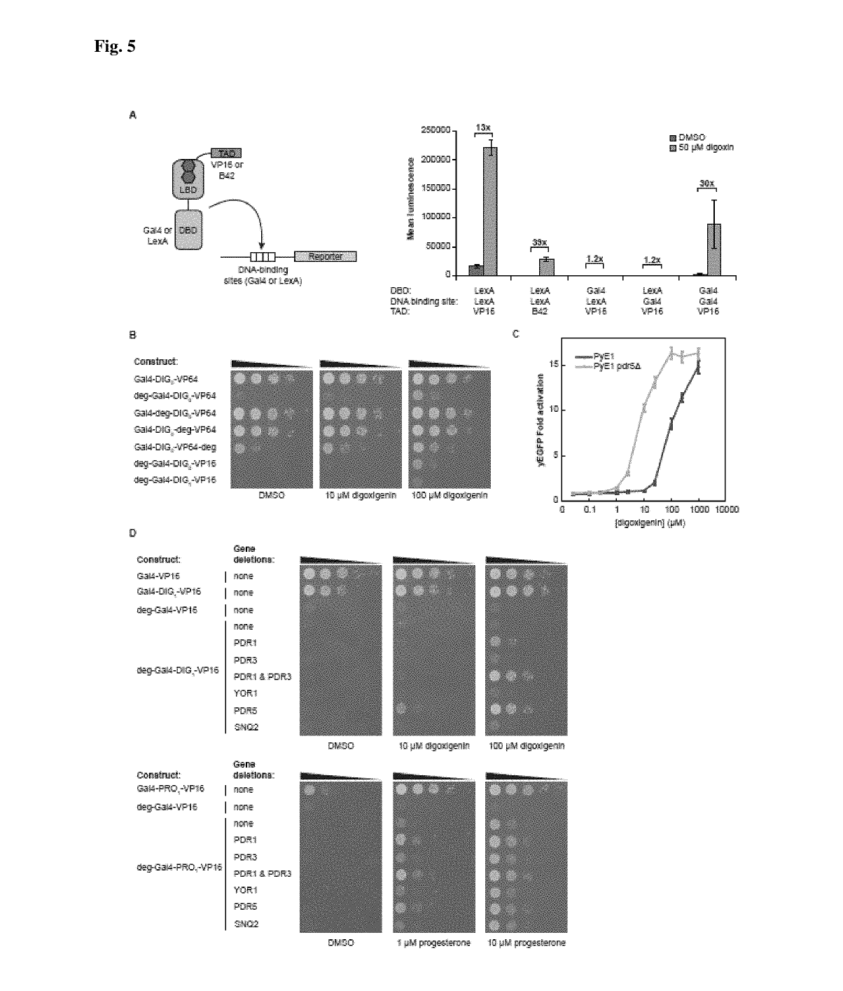

An attractive feature of the TF-biosensors is that the constituent parts—the DBD/promoter pair, the LBD, the TAD, the reporter, and the yeast strain—are modular, such that the system can be modified for additional applications. To demonstrate tunability, the DBD of G-DIG₁-V was replaced with the bacterial repressor LexA and DNA-binding sites for LexA were inserted into the GAL1 promoter. Only when the promoter driving reporter expression contained LexA-binding sites, LexA-based TF-biosensors with DIG₁, and a weak TAD, B42, produced nearly 40-fold activation in the presence of digoxin (FIG. 5a). These results demonstrate that the biosensors can function with different combinations of DBDs and TADs, which produce diverse behaviors and permit their use in eukaryotes requiring different promoters. Furthermore, the reporter gene can be swapped with an auxotrophic marker gene for growth selections. The TF biosensors drove expression of the HIS3 reporter more effectively when steroid was added to the growth media, as assessed by growth of a histidine auxotrophic strain in media lacking histidine (FIG. 5b,d,e). Fusion of the Matα2 degron to the biosensor improved dynamic range by reducing growth of yeast in the absence of ligand. Finally, the host strain could be modified to improve biosensor sensitivity toward target ligands by deletion of the gene for a multidrug efflux pump, thereby increasing ligand retention (FIG. 5c-e).

### TF-Biosensors Enable Selectable Improvements to Bioproduction of Small Molecules in Yeast

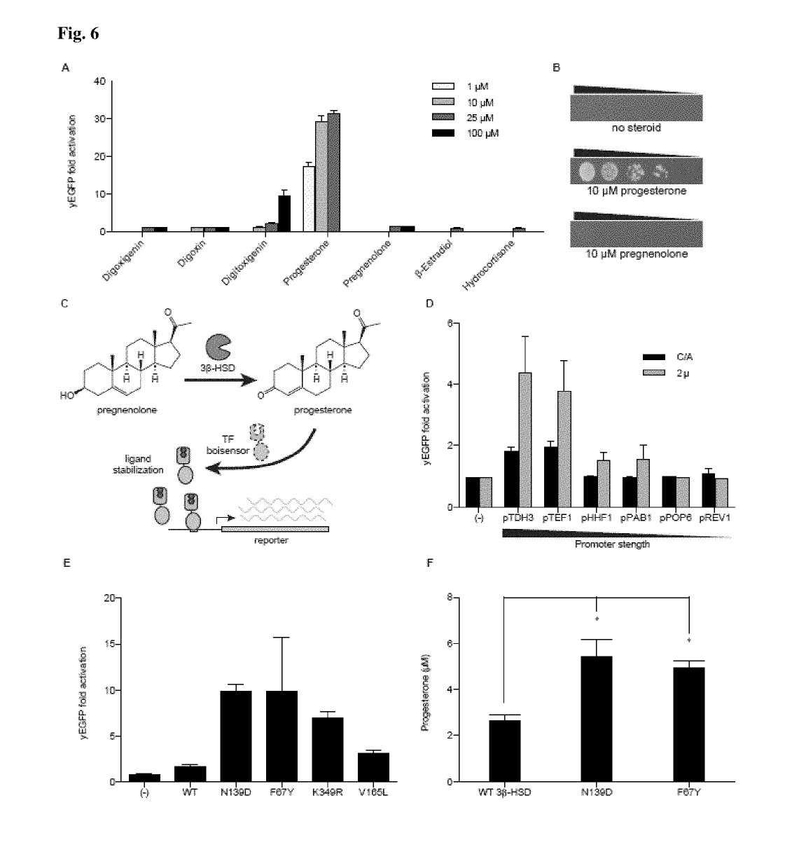

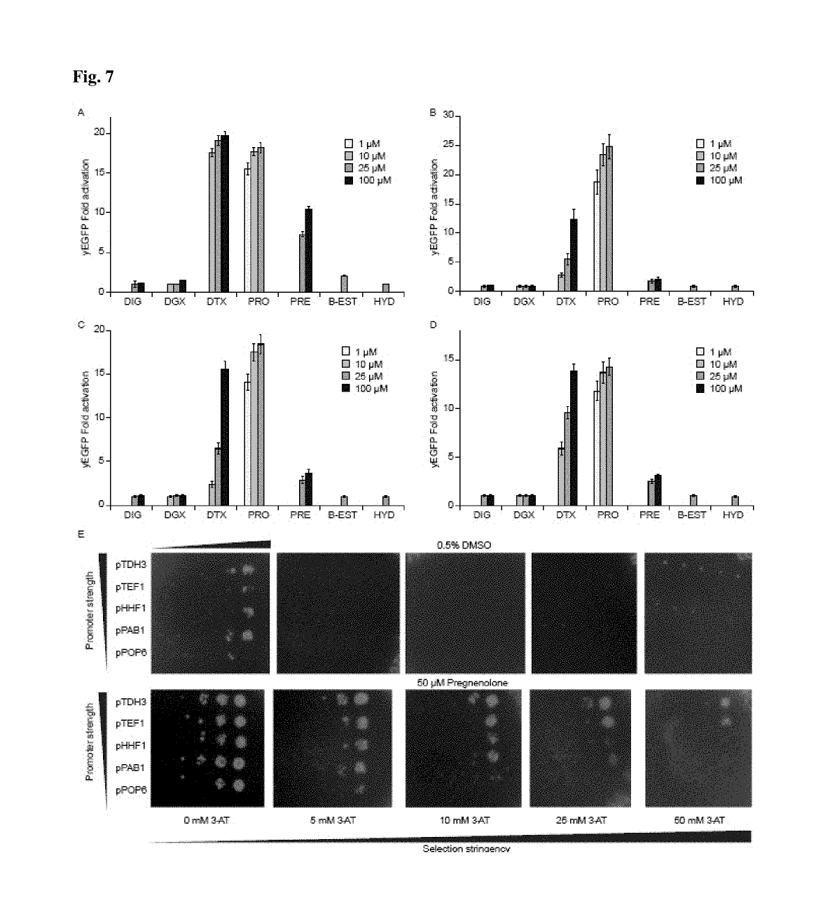

Improving bioproduction requires the ability to detect how modifications to the regulation and composition of production pathways affect product titers. Current product detection methods such as mass spectrometry or colorimetric assays are low-throughput and are not scalable or generalizable. LBD- and TF-biosensors can be coupled to fluorescent reporters to enable high throughput library screening or to selectable genes to permit rapid evolution of biosynthetic pathways. Yeast-based platforms have been developed for the biosynthesis of pharmaceutically relevant steroids, such as progesterone and hydrocortisone. A key step in the production of both steroids is the conversion of pregnenolone to progesterone by the enzyme 3β-hydroxysteroid dehydrogenase (3β-HSD). A progesterone biosensor was used to detect and improve this transformation. An important feature of biosensors intended for pathway engineering is the ability to detect a product with minimal activation by substrate or other related chemicals. TF-biosensors built from PRO₁ showed the greatest dynamic range and selectivity for progesterone over pregnenolone when driving yEGFP expression or when coupled to a HIS3 reporter assay (FIG. 6a,b and FIG. 7a). It was investigated whether this sensor could be used to detect the in vivo conversion of pregnenolone to progesterone by episomally-expressed 3β-HSD (FIG. 6c). Using G_{L77F}-PRO₁-V driving a yEGFP reporter, progesterone production was detected, with biosensor response greatest when 3β-HSD was expressed from a high copy number plasmid and from a strong promoter (FIG. 6d).

The biosensor was then used to improve this enzymatic transformation. To select for improved progesterone production, a growth assay was required in which wild-type 3β-HSD could no longer complement histidine auxotrophy when the yeast were grown on plates supplemented with pregnenolone. To this end, the selection stringency was tuned by adding the His3 inhibitor 3-aminotriazole (FIG. 7e). The 3β-HSD coding sequence was mutagenized using error-prone PCR and colonies that survived the HIS3 selection were screened for their yEGFP activation by pregnenolone. By transforming evolved 3β-HSD mutations into a fresh host background, it was shown that the mutations in the enzyme, and not off-target plasmid or host escape mutations, were responsible for increased biosensor response (FIG. 6e). Two of the mutants, 3β-HSD N139D and 3β-HSD F67Y, were assayed for progesterone production using gas chromatography and mass spectrometry and were found to produce two-fold more progesterone per OD than cells bearing the wild-type enzyme (FIG. 6f).

### Yeast-Based Biosensors Port Directly to Mammalian Cells and Tightly Regulate CRISPR/Cas9 Genome Editing

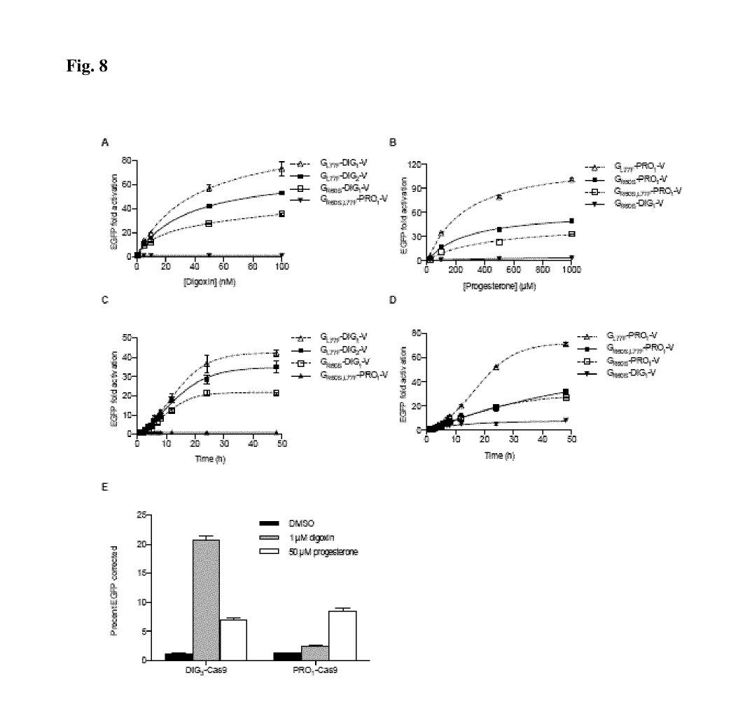

Yeast is an attractive platform for engineering in vivo biosensors because of its rapid doubling time and tractable genetics. If yeast-derived biosensors function in more complex eukaryotes, the design-build-test cycle in those organisms could be rapidly accelerated. The portability of yeast TF-biosensors to mammalian cells was first assessed. Single constructs containing digoxin and progesterone TF-biosensors with the greatest dynamic ranges (without codon optimization) were stably integrated into human K562 cells using PiggyBac transposition. The dynamics of the TF-biosensors in human cells were characterized by dose response and time course assays similar to the yeast experiments (FIG. 8a-d). As with yeast, the human cells demonstrated greater sensitivity to digoxin, with fluorescence activation peaking at 100 nM of cognate ligand for digoxin biosensors and 1 mM for progesterone biosensors. Greater than 100-fold activation was observed for the most sensitive progesterone biosensor G_{L77F}-PRO₁-V. The increase in mammalian dynamic range over yeast may arise from more aggressive degradation of destabilized biosensors or greater accumulation of target-stabilized biosensors or reporters. The time course data show that fluorescence increased four-fold within four hours of target introduction and rose logarithmically for 24-48 hours.

It was next assessed whether these biosensors could drive more complex mammalian phenotypes. The CRISPR/Cas9 system has proved to be an invaluable tool for genome editing. Despite the high programmability and specificity of Cas9-mediated gene editing achieved to date, unchecked Cas9 activity can lead to off-target mutations and cytotoxicity. Further, it may be desirable to tightly regulate Cas9 activity such that gene editing occurs only in defined conditions. To facilitate inducible gene editing, human codon-optimized versions of the DIG₃ and PRO₁ LBDs were fused to the N-terminus of Cas9 from S. pyogenes. This construct was integrated into a reporter cell line containing an EGFP variant with a premature stop codon that renders it non-functional. Upon separate stable integration of the DIG-Cas9 and PRO-Cas9 fusions, a guide RNA was transfected targeting the premature stop codon as well as a donor oligonucleotide containing the sequence to restore EGFP activity via homologous recombination. After a 48-hour incubation period, an ˜18-fold increase in GFP positive cells was observed with digoxigenin relative to the mock control (FIG. 8e).

### Environmental Detection in Plants

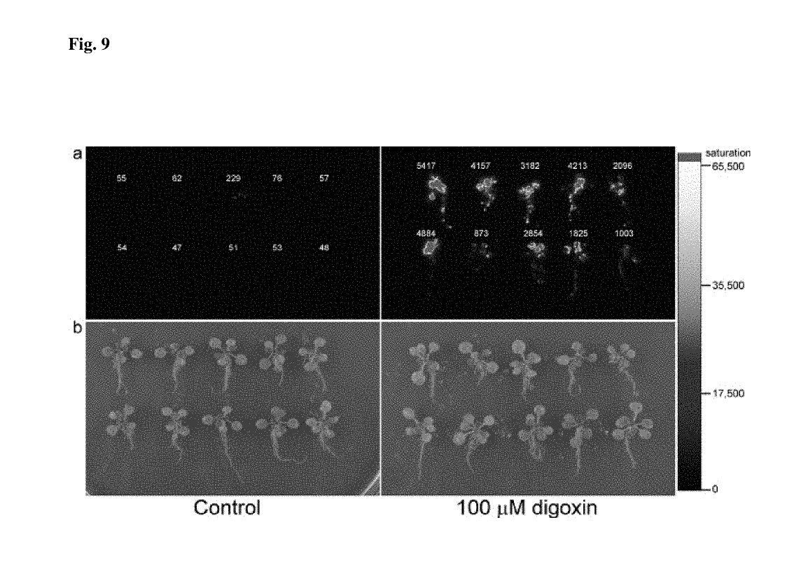

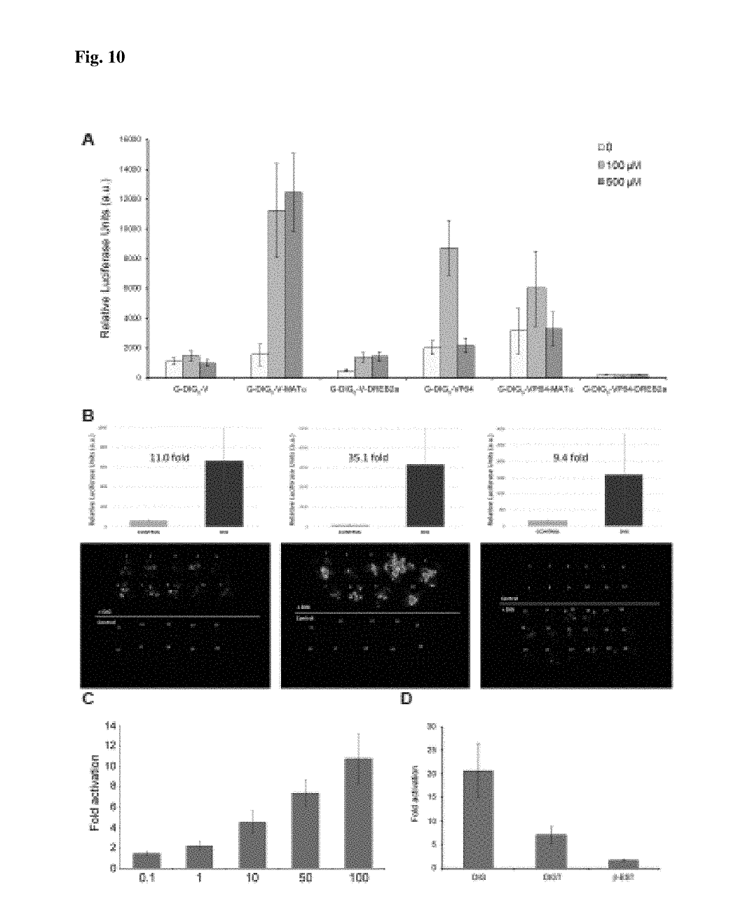

To assess generalizability of these biosensors to multicellular organisms, G-DIG₁-V was engineered to function as an environmental sensor in plants. The DIG₁ sequence was codon optimized for expression in Arabidopsis thaliana. Biosensor fusions to two different degrons, Matα2 from yeast and DREB2a from Arabidopsis, were tested with the VP16 and VP64 variants of the TAD. The G-DIG₁-TAD variants were initially tested with a transient expression assay using Arabidopsis protoplasts and a reporter gene consisting of firefly luciferase under the control of a Gal4-activated plant promoter (pUAS::Luc). The biosensor containing the Matα2 degron and VP16 TAD showed the highest fold activation of luciferase in the presence of digoxigenin (FIG. 10a). The genes encoding G-DIG₁-V-Matα2 and the Gal4-activated pUAS::Luc were next inserted into a plant transformation vector and stably transformed into Arabidopsis plants. Primary transgenic plants were screened in vivo for digoxigenin-dependent luciferase production, and responsive plants were allowed to set seed for further testing. Second generation transgenic plants (T1, heterozygous) were tested for digoxin- or digoxigen-independent induction of luciferase expression. After 42 hours, 30-50 fold induction of luciferase activity was observed in digoxigenin-treated plants compared to the uninduced control (FIG. 9).

Both digoxin and digoxigenin are capable of inducing the plant biosensor. Digoxigenin-dependent luciferase induction was observed in multiple independent transgenic T₁ lines (FIG. 10b), and an exponential dose response to digoxigenin was observed in the transgenic plants (FIG. 10c). The specificity of the digoxigenin biosensor in plants parallels that in yeast cells (FIG. 10d).

### Example 2

### Materials and Methods

Culture and Growth Conditions.

Growth media consisted of YPAD (10 g/L yeast extract, 20 g/L peptone, 40 mg/L adenine sulfate, 20 g/L glucose) and SD media (1.7 g/L yeast nitrogen base without amino acids, 5 g/L ammonium sulfate, 20 g/L glucose and the appropriate amount of dropout base with amino acids [Clontech]). The following selective agents were used when indicated: G418 (285 mg/L), pen/strep (100 U/mL penicillin and 100 ug/mL streptomycin).

LBD-yEGFP Library Construction.

The DIG10.3 sequence (Tinberg, Nature 501, 212-6, 2013) was cloned by Gibson assembly (Gibson, Nat. Methods 6, 343-345, 2009) into a pUC19 plasmid containing yeast enhanced GFP (yEGFP, UniProt ID B6UPG7) and a KanMX6 cassette flanked by 1000 and 500 bp upstream and downstream homology to the HO locus. The DIG10.3 sequence was randomized by error-prone PCR using a Genemorph II kit from Agilent Technologies. An aliquot containing 100 ng of target DNA (423 bp out of a 7.4 kb plasmid) was mixed with 5 μL of 10× Mutazyme buffer, 1 μL of 40 mM dNTPS, 1.5 μL of 20 μM forward and reverse primer containing 90 bp overlap with the pUC19 plasmid (oJF70 and oJF71), and 1 μL of Mutazyme polymerase in 50 μL. The reaction mixture was subjected to 30 cycles with Tm of 60° C. and extension time of 1 min. Vector backbone was amplified using Q5 polymerase (NEB) with oJF76 and oJF77 primers with Tm of 65° C. and extension time of 350 s. Both PCR products were isolated by 1.5% agarose gel electrophoresis and the randomized target was inserted as a genetic fusion to yEGFP by Gibson assembly. Assemblies were pooled, washed by ethanol precipitation, and resuspended in 50 μL of dH₂O, which was drop dialyzed (Millipore) and electroporated into E. coli supreme cells (Lucigen). Sanger sequencing of 16 colonies showed a mutation rate of 0-7 mutations/kb. The library was expanded in culture and maxiprepped (Qiagen) to 500 μg/μl aliquots. 16 μg of library was drop dialyzed and electrotransformed into yeast strain Y7092 for homologous recombination into the HO locus. Integrants were selected by growth on YPAD solid media containing G418 followed by outgrowth in YPAD liquid media containing G418.

LBD-yEGFP Library Selections.

Libraries of DIG₀-yEGFP and PRO₀-yEGFP integrated into yeast strain Y7092 were subject to three rounds of fluorescence activated sorting in a BD FACSAria Hu. For the first round, cells were grown overnight to an OD₆₀₀ of ˜1.0 in YPAD containing steroid (500 μM digoxigenin or 50 μM progesterone), and cells showing the top 5% of fluorescence activation were collected and expanded overnight to an OD₆₀₀ of ˜1.0 in YPAD lacking steroid. In the second sort, cells displaying the lowest ˜3% fluorescence activation were collected. Cells passing the second round were passaged overnight in YPAD containing steroid to an OD₆₀₀ of ˜1.0 and sorted once more for the upper 5% of fluorescence activation. The sorted libraries were expanded in YPAD liquid culture and plated on solid YPAD media. Ninety-six colonies from each library were clonally isolated and grown overnight in deep well plates containing 500 μL of YPAD. Candidates were diluted 1:50 into two deep well plates with SD-complete media: one plate supplemented with steroid and the other with DMSO vehicle. Cells were grown for another 4 h, and then diluted 1:3 into microtitre plates of 250 μL of the same media. Candidates were screened by analytical flow cytometry on a BD LSRFortessa cell analyzer. The forward scatter, side scatter, and yEGFP fluorescence (530 nm band pass filter) were recorded for a minimum of 20,000 events. FlowJo X software was used to analyze the flow cytometry data. The fold activation was calculated by normalizing mean yEGFP fluorescence activation for each steroid to the mean yEGFP fluorescence in the DMSO only control. Highest induction candidates were subject to Sanger sequencing with primers flanking the LBD sequence.

G-DIG-V Library Selections.

An error-prone library of G-DIG₀/DIG₁/DIG2/-V transformed into yeast strain PyE1 ΔPDR5 was subjected to three rounds of cell sorting using a Cytopeia (BD Influx) fluorescence activated cell sorter. For the first round, cells displaying high fluorescence in the presence of digoxin (on-state) were collected. Transformed cells were pelleted by centrifugation (4 min, 4000 rpm) and resuspended to a final OD₆₀₀ of 0.1 in 50 mL of SD -ura media, pen/step antibiotics, and 5 μM digoxin prepared as a 100 mM solution in DMSO. The library was incubated at 30° C. for 9 h and then sorted. Cells displaying the highest fluorescent values in the GFP channel were collected (1,747,058 cells collected of 32,067,013 analyzed; 5.5%), grown up at 30° C. in SD -ura, and passaged twice before the next sort. For the second round of sorting, cells displaying low fluorescence in the absence of digoxin (off-state) were collected. Cells were pelleted by centrifugation (4 min, 4000 rpm) and resuspended to a final OD₆₀₀ of 0.1 in 50 mL of SD -ura media supplemented with pen/strep antibiotics. The library was incubated at 30° C. for 8 h and then sorted. Cells displaying low fluorescent values in the GFP channel were collected (1,849,137 cells collected of 22,290,327 analyzed; 11.1%), grown up at 30° C. in SD -ura, and passaged twice before the next sort. For the last sorting round, cells displaying high fluorescence in the presence of digoxin (onstate) were collected. Cells were prepared as for the first sort. Cells displaying the highest fluorescent values in the GFP channel were collected (359,485 cells collected of 31,615,121 analyzed; 1.1%). After the third sort, a portion of cells were plated and grown at 30° C. Plasmids from 12 individual colonies were harvested using a Zymoprep Yeast miniprep II kit (Zymo Research Corporation, Irvine, Calif.) and the gene was amplified by 30 cycles of PCR (98° C. 10 s, 52° C. 30 s, 72° C. 40 s) using Phusion high-fidelity polymerase (NEB, Waltham, Mass.) with the T3 and T7 primers. Sanger sequencing (Genewiz, Inc., South Plainfield, N.J.) was used to sequence each clone in the forward (T3) and reverse (T7) directions.

Yeast Spotting Assays.

Yeast strain PJ69-4a transformed with p16C plasmids containing degron-G-DIG-V variants were first inoculated from colonies into SD -ura media and grown at 30° C. overnight (16 h). 1 mL of each culture was pelleted by centrifugation (3000 rcf, 2 min), resuspended in 1 mL of fresh SD -ura and the OD₆₆₀ was measured. Each culture was then diluted in SD -ura media to an OD₆₆₀=0.2 and incubated at 30° C. for 4-6 hrs. 1 mL of each culture was pelleted and resuspended in sterile, distilled water and the OD660 measured again. Each transformant was then diluted to an OD₆₆₀=0.1. Four 1/10 serial dilutions of each culture were prepared in sterile water (for a total of 5 solutions). 10 μL of each dilution was spotted in series onto several SD -ura -his agar plates containing 1 mM 3-aminotriazole and the indicated steroid. Steroid solutions were added to agar from 200× steroid solutions in DMSO (0.5% DMSO final in plates).

TF-biosensor reporter plasmid construction and integration. Reporter genes were cloned into the integrative plasmid pUG6 or the CEN plasmid pRS414 using the Gibson method50. Each reporter (either yEGFP or firefly luciferase) was cloned to include a 5′ GAL1 promoter (S. cerevisiae GAL1 ORF bases (−455)-(−5)) and a 3′ CYC1 terminator. For integration, linearized PCR cassettes containing both the reporter and an adjacent KanMX antibiotic resistance cassette were generated using primers containing 50 bp flanking sequences of homology to the URA3 locus. Integrative PCR product was transformed into the yeast strain PJ69-4a using the Gietz method54 to generate integrated reporter strains.

G-DIG/PRO-V Plasmid Construction.

G-DIG/PRO-V fusion constructs were prepared using the Gibson method (PMID 19363495). Constructs were cloned into the plasmid p416CYC (p16C). Gal4 (residues 1-93, UniProt ID P04386), DIG10.3 (PMID 24005320), and VP16 (residues 363-490, UniProt ID P06492) PCR products for were amplified from their respective templates using Phusion high-fidelity polymerase (NEB, Waltham, Mass.) and standard PCR conditions (98° C. 10 s, 60° C. 20 s, 72° C. 30 s; 30 cycles). An 8-residue linker sequence (SEQ ID NO:1) was used between Gal4 and DIG10.3. PCR primers were purchased from Integrated DNA technologies and contained 24-30 5′ bases of homology to either neighboring fragments or plasmid. Clones containing an N-terminal degron were similarly cloned fusing residues 1-67 of Mat-alpha2 (UniProt ID P0CY08) to the 5′-end of G-DIG-V. Plasmids were transformed into yeast using the Gietz method (PMID 17401334), with transformants being plated on synthetic complete media lacking uracil (SD -ura).

G-DIG-V Mutant Construction.

Mutations were introduced into DIG10.3/pETCON14 or the appropriate G-DIG/PRO-V construct using Kunkel mutagenesis (Kunkel, PNAS USA, 82, 488-492, 1985). Oligos were ordered from Integrated DNA Technologies, Inc. For mutants constructed in pETCON/DIG10.3, the mutagenized DIG10.3 gene was amplified by 30 cycles of PCR (98° C. 10 s, 61° C. 30 s, 72° C. 15 s) using Phusion high-fidelity polymerase (NEB, Waltham, Mass.) and 5′- and 3′-primers having homologous overlap with the DIG10.3-flanking regions in p16C-G-DIG-VP64. Genes were inserted into p16C-Gal4-(HE)-VP16 by Gibson assembly 50 using vector digested with HindIII and EcoRI-HF.

G-PRO-V Mutant Construction.

The genes for DIG10.3 Y34F/Y99F/Y101F were amplified from the appropriate DIG10.3/pETCON (PMID 24005320) construct by 30 cycles of PCR (98° C. 10 s, 59° C. 30 s, 72° C. 15 s) using Phusion high-fidelity polymerase (NEB, Waltham, Mass.) and 5′- and 3′-primers having homologous overlap with the DIG10.3-flanking regions in p16CG-DIG-VP64. Genes were inserted into p16C-GDVP16 by Gibson assembly50 using p16C-Gal4-(HE)-VP16 vector digested with HindIII and EcoRI-HF.

G-DIG-V Error-Prone Library Construction.

A randomized G-DIG-V library was constructed by error-prone PCR using a Genemorph II kit from Agilent Technologies. An aliquot containing 20 ng p16C GDVP16, 20 ng p16C GDVP16 E83V, and 20 ng p16C Y36H was mixed with 5 μL of 10× Mutazyme buffer, 1 μL of 40 mM dNTPS, 1.5 μL of 20 μM forward and reverse primer containing 37- and 42-bp overlap with the p16C vector for homologous recombination, respectively, and 1 μL of Mutazyme polymerase in 50 μL. The reaction mixture was subjected to 30 cycles of PCR (95° C. 30 s, 61° C. 30 s, 72° C. 80 s). Template plasmid was digested by adding 1 μL of DpnI to the reaction mixture and incubating for 3 hr at 37° C. Resulting PCR product was purified using a Quiagen PCR cleanup kit, and a second round of PCR was used to amplify enough DNA for transformation. Gene product was amplified by combining 100 ng of mutated template DNA with 2.5 μL of 10 μM primers, 10 μL of 5× Phusion buffer HF, 1.5 μL of DMSO, and 1 μL of Phusion high-fidelity polymerase (NEB, Waltham, Mass.) in 50 μL. Product was assembled by 30 cycles of PCR (98° C. 10 s, 65° C. 30 s, 72° C. 35 s). Following confirmation of a single band at the correct molecular weight by 1% agarose gel electrophoresis, the PCR product was purified using a Quiagen PCR cleanup kit and eluted in ddH2O. Yeast strain PyE1 ΔPDR5 was transformed with 9 μg of amplified PCR library and 3 μg of p16C Gal4-(HE)-VP16 triply digested with SalI-HF, BamHI-HR, and EcoRI-HF using the method of Benatuil56, yielding ˜106 transformants. Following transformation, cells were grown in 150 mL of SD -ura media. Sanger sequencing of 12 individual colonies revealed an error rate of ˜1-6 mutations per gene.

G-DIG-V Error-Prone Library Mutation Screens.

Of twelve sequenced clones from the library sorts, two showed significantly improved (>2-fold) response to DIG over the input clones (clone 3 and clone 6). Clone 3 contains the following mutations: Gal4_T44T (silent), Gal4_L77F, DIG10.3_E5D, DIG10.3_E83V, DIG10.3_R108R (silent), DIG10.3_L128P, DIG10.3_I137N, DIG10.3_S143G, and VP16_A44T. Clone 6 contains the following mutations: Gal4_R60S, Gal4_L84L (silent), VP16_G17G (silent), VP16_L48V, and VP16_H98H (silent). To identify which mutations led to the observed changes in DIG response, variants of these clones with no silent mutations and each individual point mutant were constructed using Kunkel mutagenesis.

Oligos were ordered from Integrated DNA Technologies, Inc. Sequence-confirmed plasmids were transformed into PyE1 ΔPDR5f and plated onto selective SD -ura media. Individual colonies were inoculated into liquid media, grown at 30° C., and passaged once. Cells were pelleted by centrifugation (4 min, 1700×g) and resuspended to a final OD660 of 0.1 in 1 mL of SD -ura media supplemented 50 μM DIG prepared as a 100 mM solution in DMSO. Following a 6 hr incubation at 30° C., cells were pelleted, resuspended in 200 μL of PBS, and cellular fluorescence was measured on an Accuri C6 flow cytometer using a 488 nm laser for excitation and a 575 nm band pass filter for emission. FlowJo software version 7.6 was used to analyze the flow cytometry data. Data are given as the mean yEGFP fluorescence of the single yeast population in the absence of DIG (off-state) and the mean yEGFP fluorescence of the higher fluorescing yeast population in the presence of DIG (on-state).

Computational Model of Gal4-DIG.

A model of the Gal4-DIG10.3 fusion was built using Rosetta Remodel (PMID 21909381) to assess whether the linker between Gal4 and the DIG LBD, which are both dimers, would allow for the formation of a dimer in the fusion construct. In the simulation, the Gal4 dimer was held fixed while the relative orientation of the DIG LBD monomers were sampled symmetrically using fragment insertion in the linker region. Constraints were added across the DIG LBD dimer interface to facilitate sampling. The lowest energy model satisfied the dimer constraints, indicating that a homodimer configuration of the fusion is possible.

TF-Biosensor Titration Assays in Yeast.

Yeast strain PyE1 transformed with p16C plasmids containing G-LBD-V variants were inoculated from colonies into SD -ura media supplemented and grown at 30° C. overnight (16 h). 10 μL of the culture was resuspended into 490 μL of separately prepared media each containing a steroid of interest (SD -ura media supplemented the steroid of interest and DMSO to a final concentration of 1% DMSO). Resuspended cultures were then incubated at 30° C. for 8 hours. 125 μL of incubated culture was resuspended into 150 μL of fresh SD -ura media supplemented with the steroid of interest and DMSO to a final concentration of 1%. These cultures were then assayed by analytical flow cytometry on a BD LSRFortessa using a 488 nm laser for excitation. The forward scatter, side scatter, and yEGFP fluorescence (530 nm band pass filter) were recorded for a minimum of 20,000 events. FlowJo X software was used to analyze the flow cytometry data. The fold activation was calculated by normalizing mean yEGFP fluorescence activation for each steroid to the mean yEGFP fluorescence in the DMSO only control. G-PRO₀-V was assayed on a separate day from the other TF biosensors under identical conditions.

TF-Biosensor Kinetic Assays in Yeast.

Yeast strain PyE1 transformed with p16C plasmids containing G-LBD-V variants were inoculated from colonies into SD -ura media and grown at 30° C. overnight (16 h). 5 μL of each strain was diluted into 490 μL of SD -ura media in 2.2 mL plates. Cells were incubated at 30° C. for 8 hours. 5 μL of steroid was then added for a final concentration of 250 μM digoxin or 50 μM progesterone. For each time point, strains were diluted 1:3 into microtitre plates of 250 μL of the same media. Strains were screened by analytical flow cytometry on a BD LSRFortessa cell analyzer. The forward scatter, side scatter, and yEGFP fluorescence (530 nm band pass filter) were recorded for a minimum of 20,000 events. FlowJo X software was used to analyze the flow cytometry data. The fold activation was calculated by normalizing mean yEGFP fluorescence activation for each time point to the mean yEGFP fluorescence at T=0 h.

Luciferase Reporter Assay.

Yeast strains containing either a plasmid-borne or integrated luciferase reporter were transformed with p16C plasmids encoding TF-biosensors. Transformants were grown in triplicate overnight at 30° C. in SD -ura media containing 2% glucose in sterile glass test tubes on a roller drum. After ˜16 hours of growth, OD600 of each sample was measured and cultures were back diluted to OD600=0.2 in fresh SD -ura media containing steroid dissolved in DMSO or a DMSO control (1% DMSO final). Cultures were grown at 30° C. on roller drum for 8 hrs prior to taking readings. Measurement of luciferase activity was adapted from a previously reported protocol58. 100 uL of each culture was transferred to a 96-well white NUNC plate. 100 uL of 2 mM D-luciferin in 0.1 M sodium citrate (pH 4.5) was added to each well of the plate and luminescence was measured on a Victor 3V after 5 minutes. Yeast deletion strain creation. Genomic deletions were introduced into the yeast strains PJ69-4a and PyE1 using the 50:50 method57. Briefly, forward and reverse primers were used to amplify an URA3 cassette by PCR. These primers generated a product containing two 50 bp sequences homologous to the 5′ and 3′ ends of the ORF at one end and a single 50 bp sequence homologous to the middle of the ORF at the other end. PCR products were transformed into yeast using the Gietz method54 and integrants were selected on SD -ura plates. After integration at the correct locus was confirmed by a PCR screen, single integrants were grown for 2 days in YEP containing 2.5% ethanol and 2% glycerol. Each culture was plated on synthetic complete plates containing 5-fluoroorotic acid. Colonies were screened for deletion of the ORF and elimination of the Ura3 cassette by PCR and confirmed by DNA sequencing.

TF-Biosensor Specificity Assays.

Yeast strains expressing the TF-biosensors and yEGFP reporter (either genetically fused or able to be transcriptionally activated by the TAD) were grown overnight at 30° C. in SD -ura media for 12 hours. Following overnight growth, cells were pelleted by centrifugation (5 min, 5250 rpm) and resuspended into 500 μL of SD -ura. 10 μL of the washed culture was resuspended into 490 μL of separately prepared media each containing a steroid of interest (SD -ura media supplemented with the steroid of interest and DMSO to a final concentration of 1% DMSO). Steroids were tested at a concentration of 100 μM digoxin, 50 μM progesterone, 250 μM pregnenolone, 100 μM digitoxigenin, 100 μM beta-estradiol, and 100 μM hydrocortisone. Stock solutions of steroids were prepared as a 50 mM solution in DMSO. Resuspended cultures were then incubated at 30° C. for 8 hours. 125 μL of incubated culture was resuspended into 150 μL of fresh SD -ura media supplemented the steroid of interest, and DMSO to a final concentration of 1%. These cultures were then assayed by analytical flow cytometry on a BD LSRFortessa using a 488 nm laser for excitation. The forward scatter, side scatter, and yEGFP fluorescence (530 nm band pass filter) were recorded for a minimum of 20,000 events. FlowJo X software was used to analyze the flow cytometry data. The fold induction was calculated by normalizing mean yEGFP fluorescence activation for each steroid to the mean yEGFP fluorescence in the DMSO only control.

3β-HSD Plasmid and Library Construction.

The 3β-HSD ORF was synthesized as double stranded DNA (Integrated DNA Technologies, Inc.) and amplified using primers oJF325 and oJF326 using KAPA HiFi under standard PCR conditions and digested with BsmBI to create plasmid pJF57. 3β-HSD expression plasmids (pJF76 through pJF87) were generated by digesting plasmid pJF57 along with corresponding plasmids from the Yeast Cloning Toolkit59 with BsaI and assembled using the Golden Gate Assembly method (Engler, et al. PLoS One 3, 2008). The 3β-HSD sequence was randomized by error-prone PCR using a Genemorph II kit from Agilent Technologies. An aliquot containing 100 ng of target DNA was mixed with 5 μL of 10× Mutazyme buffer, 1 μL of 40 mM dNTPS, 1.5 μL of 20 μM forward and reverse primer containing 90-bp overlap with the 3β-HSD expression plasmids and 1 μL of Mutazyme polymerase in 50 μL. The reaction mixture was subject to 30 cycles with Tm of 60° C. and extension time of 1 min. Vector backbone was amplified using KAPA HiFi polymerase with oJF387 and oJF389 (pPAB1) or oJF387 and oJF389 (pPOP6) with Tm of 65° C. and extension time of 350 s. PCR products were isolated by 1.5% agarose gel electrophoresis and assembled using the Gibson method 50. Assemblies were pooled, washed by ethanol precipitation, and resuspended in 50 μL of dH2O, which was drop dialyzed (Millipore) and electroporated into E. cloni supreme cells (Lucigen). Sanger sequencing of 16 colonies showed a mutation rate of 0-4 mutations/kb. The library was expanded in culture and maxiprepped (Qiagen) to 500 μg/μL aliquots. 16 μg of library was drop dialyzed and electrotransformed into yeast strain PyE1.

3β-HSD Progesterone Selections.

PyE1 transformed with libraries of 3β-HSD were seeded into 5 mL of SD -ura -leu media supplemented and grown at 30° C. overnight (24 h). Cultures were measured for OD₆₀₀, diluted to an OD₆₀₀ of 0.0032, and 100 μL was plated onto SD -ura -leu -his plates supplemented 35 mM 3-AT and either 50 μM pregnenolone or 0.5% DMSO.

Progesterone Bioproduction and GC/MS Analysis.

Production strains were inoculated from colonies into 5 mL SD -ura media and grown at 30° C. overnight (24 h). 1 mL of each culture was washed and resuspended into 50 mL of SD -ura with 250 mM of pregnenolone and grown at 30° C. for 76 h. OD₆₀₀ measurements were recorded for each culture before pelleting by centrifugation. Cells were lysed by glass bead disruption, and lysates and growth media were extracted separately with heptane. Extractions were analyzed by GC/MS.

TF-Biosensor EGFP Assays in Mammalian Cells.

For each TF-biosensor, 1 μg of the PiggyBac construct along with 400 ng of transposase were nucleofected into K562 cells using the Lonza Nucleofection system as per manufacturer settings. Two days post-transfection, cells underwent puromycin selection (2 μg/mL) for at least eight additional days to allow for unintegrated plasmid to dilute out and ensure that all cells contained the integrated construct. An aliquot of 100,000 cells of each integrated population were then cultured with 25 μM of progesterone, 1 μM of digoxigenin, or no small molecule. Forty-eight hours after small molecule addition, cells were analyzed by flow cytometry using a BD Biosciences Fortessa system. Mean EGFP fluorescence of the populations was compared.

Construction of K562 Cell Lines.

The PiggyBac transposase system was employed to integrate biosensor constructs into K562 cells. Vector PB713B-1 (Systems Biosciences) was used a backbone. Briefly, this backbone was digested with NotI and HpaI and G-LBD-V, Gal4BS-E1b-EGFP (EGFP; enhanced GFP ref or UniProt ID A0A076FL24), and sEF1-Puromycin were cloned in. Gal4BS represents four copies of the binding sequence. For hCas9, the PiggyBac system was also employed, but the biosensors were directly fused to the N-terminus of Cas9 and were under control of the CAGGS promoter. Cas9 from S. pyogenes was used.

TF-Biosensor-Cas9 Assays.

Construct integration was carried out as for the Cas9 experiments for EGFP assays, except that the constructs were integrated into K562 containing a broken EGFP reporter construct. Introduction of an engineered nuclease along with a donor oligonucleotide can correct the EGFP and produce fluorescent cells. Upon successful integration (˜10 days after initial transfection), 500,000 cells were nucleofected with 500 ng of guide RNA (sgRNA) and 2 μg of donor oligonucleotide. Nucleofected cells were then collected with 200 μL of media and 50 μL aliquots were added to wells containing 950 μL of media. Each nucleofection was split into four separate wells containing 1 μM of digoxigenin, 25 μM of progesterone, or no small molecule. Forty-eight hours later, cells were analyzed using flow cytometry and the percentage of EGFP positive cells was determined.

TF-Biosensor Assays in Protoplasts.

Digoxin transcriptional activators were initially tested in a transient expression assay using Arabidopsis protoplasts according previously described methods (Yoo, et al. Nat. Protoc. 2, 1565-1572, 2007), with some modifications. Briefly, protoplasts were prepared from 6-week old Arabidopsis leaves excised from plants grown in short days. Cellulase Onozuka R-10 and Macerozyme R-10 (Yakult Honsha, Inc., Japan) in buffered solution were used to remove the cell wall. After two washes in W5 solution, protoplasts were re-suspended in MMg solution at 2×105 cells/mL for transformation. Approximately 104 protoplasts were mixed with 5 mg of plasmid DNA and PEG4000 at a final concentration of 20%, and allowed to incubate at room temperature for 30 minutes. The transformation reaction was stopped by addition of 2 volumes of W5 solution, and after centrifugation, protoplasts were re-suspended in 200 mL of WI solution (at 5×105/mL) and plated in a 96-well plate. Digoxigenin (Sigma-Aldrich, St. Louis, Mo.) was added to the wells, and protoplasts were incubated overnight at room temperature in the dark, with slight shaking (40 rpm). For luciferase imaging, protoplasts were lysed using Passive Lysis Buffer (Promega, Madison, Wis.) and mixed with LARII substrate (Dual-Luciferase Reporter Assay System, Promega). Luciferase luminescence was collected by a Stanford Photonics XR/MEGA-10 ICCD Camera and quantified using Piper Control (v.2.6.17) software.

Plant Plasmid Construction.

G-DIG₁-V was recoded to function as a ligand-dependent transcriptional activator in plants. Specifically, an Arabidopsis thaliana codon optimized protein degradation sequence from the yeast MATα gene was fused in frame in between the Gal4 DBD and the DIG₁ LBD. The resulting gene sequence was codon-optimized for optimal expression in Arabidopsis thaliana plants and cloned downstream of a plant-functional CaMV35S promoter to drive constitutive expression in plants, and upstream of the octopine synthase (ocs) transcriptional terminator sequence. To quantify the transcriptional activation function of DIG10.3, the luciferase gene from Photinus pyralis (firefly) was placed downstream of a synthetic plant promoter consisting of five tandem copies of a Gal4 Upstream Activating Sequence (UAS) fused to the minimal (−46) CaMV35S promoter sequence. Transcription of luciferase is terminated by the E9 terminator sequence. These sequences were cloned into a pJ204 plasmid and used for transient expression assays in Arabidopsis protoplasts.

Construction of Transgenic Arabidopsis Plants.

After confirmation of function in transient tests, the digoxin biosensor genetic circuit was transferred to pCAMBIA 2300 and was stably transformed into Arabidopsis thaliana ecotype Columbia plants using a standard Agrobacterium floral dip method. Transgenic plants were selected in MS media 53 containing 100 mg/L kanamycin.

TF-Biosensor Assays in Transgenic Plants.

Transgenic plants expressing the digoxin biosensor genetic circuit were tested for digoxigenin-induced luciferase expression by placing 14-16 day old plants in liquid MS (-sucrose) media supplemented with 0.1 mM digoxigenin in 24-well plates, and incubated in a growth chamber at 24° C., 100 mE·m²·s⁻¹ light. Luciferase expression was measured by imaging plants with a Stanford Photonics XR/MEGA-10 ICCD Camera, after spraying luciferin and dark adapting plants for 30 minutes. Luciferase expression was quantified using Piper Control (v.2.6.17) software. Plants from line KJM58-10 were used to test for specificity of induction by incubating plants, as described above, in 0.1 mM digoxigenin, 0.1 mM digitoxigenin, and 0.02 mM β-estradiol. All chemicals were obtained from Sigma-Aldrich (St. Louis, Mo.).

### Example 3

### Design of Fentanyl Binders

Fentanyl is a potent agonist of the μ-opioid receptor, with an affinity of approximately 1 nM and a potency 100-times that of morphine. It is used both pre- and post-operatively as a pain management agent. The fast acting nature and strength of fentanyl have been attributed to its high degree of lipophilicity. Recently, fentanyl has become a widespread recreational drug of abuse, with increasing reports of illegal manufacturing and fentanyl-related deaths across the country and other parts of the world.

Fentanyl binders were designed using a two-step approach. Fentanyl contains 6 rotatable bonds, which increases the combinatorial complexity of possible protein-ligand interactions to be considered. Starting from the structure of a fentanyl-citrate toluene solvate (Peeters et al., 1979), 11 conformers plus an additional hydrated model of fentanyl were generated based on the small molecule structure, with non-covalently bound water atoms at both the tertiary amine (3 Å nitrogen to water distance, 109° carbon-nitrogen-water angle) and the carbonyl oxygen (3 Å oxygen to water distance, 120° carbon-oxygen-water angle). For each fentanyl conformer, a large number of shape complementary placements of fentanyl within protein scaffolds from the MOAD database was identified (Hu, Proteins 60:3, pp. 333-340, 2005) using the fast docking algorithm PatchDock, which identifies shape complementary interactions between binding partners (Duhovny et al., 2002).

In the second design step, the top 20 scoring docks from PatchDock for each scaffold were selected and optimized the identities and rotamer conformations of amino acids within 8 Å of fentanyl for shape complementarity and specific protein-ligand interactions. Similar to other μ-opioid receptor agonists, fentanyl possesses a charged tertiary amine, one of only two sites capable of making electrostatic interactions. The tertiary amine was exploited to confer directionality and allow atomic level control over the placement of the otherwise hydrophobic molecule. Two design strategies were pursued: 1) The introduction of specific side chain-fentanyl interactions, either acidic (Asp or Glu) or cation-pi (Phe, Tyr, Trp) with the tertiary amine, and 2) the use of the hydrated fentanyl for bridging indirect fentanyl-protein interactions. Designs were filtered based on shape complementarity, protein-fentanyl interface energy and the solvent-accessible-surface-area (SASA), and 62 were selected for experimental characterization.

The designs were expressed on the yeast surface and probed for binding with a bovine serum albumin-fentanyl (Fen-BSA) conjugate. Sixty-one of the 62 designs expressed well, and 3 bound fentanyl with low micromolar to high nanomolar affinities. Fen49, the strongest binder (500 nM affinity for Fen-BSA) on yeast (SEQ ID NO: 23), and Fen21 (10 μM; SEQ ID NO: 21) were chosen for further experimental characterization, as they represent two different scaffold classes. Of these two designs, recombinantly expressed Fen49 proved to be more stable and amenable to crystallization. Purified Fen49 displayed an affinity of 6.9 μM for a fentanyl-Alexa-488 conjugate by fluorescence polarization. Fentanyl does not have an affinity for the unmodified scaffold (FIG. 2A), a glycoside hydrolase (PDB 2QZ3). Following the placement of the hydrated fentanyl into the binding pocket via PatchDock, RosettaDesign introduced 9 mutations to 2QZ3 in order to optimize the protein-ligand interactions. Yeast binding experiments of individual Fen49 point mutants corresponding to the computationally substituted positions showed that the majority are crucial for recognizing fentanyl (FIG. 2B). 2QZ3 was cocrystallized with xylotetraose (only 3 of the 4 xylose molecules were placed in the final 2QZ3 model), a sugar molecule with a high degree of polarity compared with fentanyl (FIG. 1B) (Vandermarliere et al., 2008). Such a dramatic repurposing of a sugar binding protein is possible because the initial low-resolution docking step is agnostic to the polar character of the scaffold binding cavity, as shape complementarity is the primary focus.

An atomic resolution (1.00 Å) X-ray crystal structure of Fen49 in the apo state was solved, one of the first examples of an original (non-optimized) computational design that has been structurally characterized. The structure revealed a highly preorganized binding cavity (28 of 30 non-alanine/non-glycine side chains within ˜8 Å of fentanyl adopt the designed rotamer) and an overall structure in very close agreement with the design model; the RMSD of the design model to the parent structure is 0.26 over 184 of 185 residues (TM_align score of 0.99). The Fen49 apo crystals were obtained from a condition containing 25% polyethylene glycol (PEG) 3350 as the precipitant. During model building, a well ordered portion of PEG was observed in the binding cavity. Soaking experiments with fentanyl tended to crack the crystals and destroy X-ray diffraction, likely as a result of PEG being displaced from the binding cavity. This fact, coupled with a lack of alternate crystal forms, prevented obtaining a structure of the parent Fen49-fentanyl complex.

In order to obtain a detailed map of the sequence determinants of folding and binding, site-saturation mutagenesis (SSM) was carried out on 184 of the 185 Fen49 residues, with the exception of the start methionine. Next-gen sequencing was carried out after each of 4 rounds of affinity enrichment. The majority of the binding site residues were preserved during selection, suggesting that Fen49 was designed with a near-optimal binding cavity. Exceptions to this were three alanine residues, A67, A78 and A172, at the base of the binding pocket that were frequently substituted with larger hydrophobic residues, which would likely provide additional packing for fentanyl. Two positions above the binding cavity enriched to amino acids that could reduce steric hindrance (Arg 112 to smaller aliphatic amino acids) or function as a hydrophobic lid over the binding site (Pro 116 to larger side chains). Charged amino acids, which might be expected to destabilize the hydrophobic cavity of Fen49, were disfavored during selection. However, a modest enrichment for glutamate at position 37 was observed in the second round of selection, suggesting an E37-tertiary amine salt bridge and the possibility of alternative poses of fentanyl within the binding site. This substitution was depleted in later rounds as a hydrophobic pocket was ultimately selected. A specific combination of 2 substitutions, A78V plus A172I, were identified that produced a Fen49 variant with an approximate 100-fold affinity improvement for fentanyl, to 64 nM. These substitutions increase packing in the binding site, and likely require a modest positional adjustment of fentanyl in order to avoid a steric clash with I172.

From the SSM experiments, a Fen49 Y88A point mutant was identified, termed Fen49*, that proved to be more suitable for complex structure determination. The 1.79 Å Fen49*-apo structure again revealed a highly preorganized binding site, and an overall structure in close agreement with the Fen49 design (0.72 RMSD for Fen49* compared with the design model over 184 of 185 residues (TM_align score of 0.98)). The majority of Fen49* side chains adopt the design conformations (25 of 30 non-alanine/non-glycine residues within ˜8 Å of fentanyl are correct) and the structure shows minimal backbone rearrangements. The only significant deviation from the parent Fen49 is observed in the loop region Thr87-Thr93, which contains the Y88A substitution (FIG. 3 and fig. S5). In addition, a 3-residue polar network between Arg89, Asp106 and Tyr108 on the backside of the binding cavity is disrupted in Fen49* (fig. S6). As a consequence of the altered loop, tryptophans 63 and 90 adopt non-designed rotamers and collapse inwards towards the center of the binding cavity, with the designed Trp90-fentanyl stacking interaction replaced by a Trp63-fentanyl dipole-quadrupole.

Unlike the parent Fen49, Fen49*-apo produced crystals with an empty binding cavity that proved to be useful for soaking experiments. A 1.67 Å Fen49*-fentanyl complex structure was solved, which again exhibited a high degree of similarity compared both with the designed model (RMSD of 0.64 over 184/185 residues, TM_align score of 0.99), as well as with the Fen49*-apo structure (RMSD of 0.420 over all 185 residues, TM_align score of 0.99). The Thr87-Thr93 loop adopts the same structure as that found in Fen49*-apo. With the exception of Trp63, which is flipped nearly 180° in the complex, fentanyl does not induce any significant changes to the active site upon binding. Fentanyl appears to stabilize the binding site; Fen49*-apo Trp63 and the Thr87-Thr93 loop exhibit higher than average B-factors when compared both with the Fen49*-apo structure overall and with the corresponding residues in the Fen49* complex. Despite the divergent Thr87-Thr93 loop, the parent Fen49 and Fen49* have virtually identical affinities for fentanyl, suggesting that this loop, and more specifically the differential Trp63-90 interaction with fentanyl, do not substantially lower the free energy of fentanyl binding. Instead, preorganization of the inner binding cavity residues appears to be the main determinant for binding.

Fen49 was designed to bind a solvated fentanyl. The water modeled at the fentanyl tertiary amine was introduced in order to bridge an indirect protein-ligand interaction with Tyr80. During structure refinement, a strong electron density peak was observed at this location (3 Å distance and 109.2° angle). Refinement with water at this position produced a strong positive signal in the Fo−Fc difference map, and it became clear that the density corresponded instead to a chloride ion. This chloride functions as a surrogate to the designed water; it is coordinated by the tertiary amine, Tyr80 and a nearby water, a trigonal planar arrangement for chloride typically found in the PDB (Carugo et al., 2014). The Tyr80-chloride interaction observed in Fen49* is mimicked by a Tyr80-PEG bond in the Fen49 parent structure (fig. S2). A second water molecule was observed bound to the fentanyl carbonyl oxygen at the designed position (2.7 Å distance, 135.2° angle).

### Example 4

### Biosensors for Fentanyl Detection

Fentanyl detectors were developed by incorporating fentanyl binders Fen21 and Fen49 into the transcription factor (TF)-based biosensor system. The Fen49 and Fen21 transcription factors were engineered by N-terminal fusion of the yeast MATα gene degron and the Gal4 DNA binding domain and C-terminal fusion of the VP16 transcriptional activator to either Fen49 or Fen21. The resulting gene sequence was codon-optimized for optimal expression in Arabidopsis thaliana plants and cloned downstream of the CaMV35S promoter to drive constitutive expression in plants, and upstream of the octopine synthase (ocs) transcriptional terminator sequence. To quantify the transcriptional activation function of the Fen49 and Fen21 transcription factors, the luciferase gene from Photinus pyralis (firefly) was placed downstream of a synthetic plant promoter consisting of five tandem copies of a Gal4 Upstream Activating Sequence (UAS) fused to the minimal (−46) CaMV35S promoter sequence. Transcription of luciferase is terminated by the E9 terminator sequence. These sequences were cloned into a pSEVA 141 plasmid and used for transient expression assays in Arabidopsis protoplasts.

The construct for Fen21 transcription and luciferase reporting was inserted into the pCAMBIA 2300 plant transformation vector and stably transformed into Arabidopsis thaliana ecotype Columbia plants using a standard Agrobacterium tumefaciens floral dip protocol. Primary transgenic plants were screened in vivo for fentanyl-dependent luciferase production using a Stanford Photonics XR/MEGA-10Z ICCD Camera and Piper Control Software System, and responsive plants were allowed to set seed for further testing. Second generation transgenic plants (T₁, heterozygous) were tested for fentanyl-dependent induction of luciferase expression.

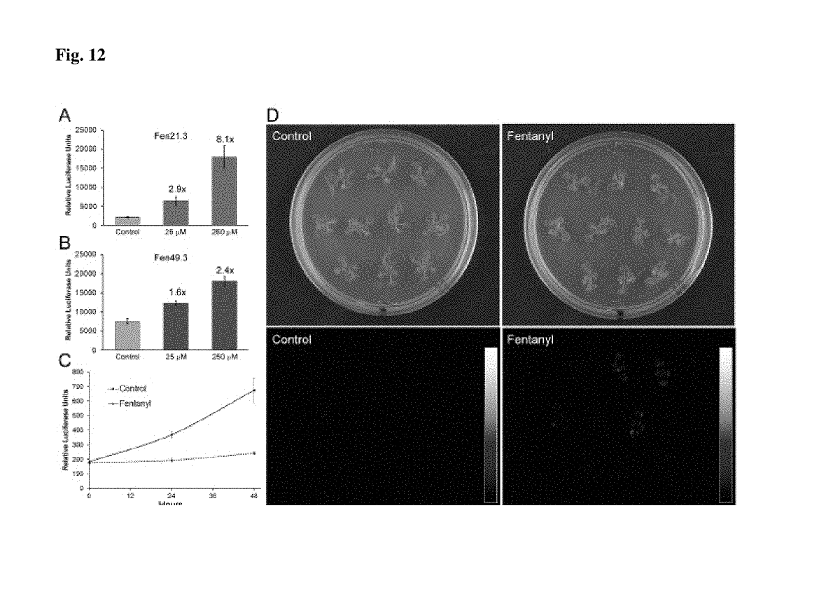

Using firefly luciferase under the control of the Gal4-activated plant promoter as a readout in Arabidopsis thaliana protoplasts, unmodified binders were shown to be effective for the TF-based approach (FIG. 12a, b). Fen21 proved to be the more responsive sensor, showing an 8-fold increase in luciferase expression over background when treated with 250 μM fentanyl in plant protoplasts. Fen49 expressing protoplasts showed a 2.4-fold increase in luciferase expression. Next, Arabidopsis plants were stably transformed and it was found that whole plants are also responsive to fentanyl (FIG. 12c,d). Continuous exposure of heterozygous (T₁) transgenic plants to 500 μM fentanyl resulted in ˜3.7-fold induction of luciferase expression after 48 hours.

What is claimed is:

1. A recombinant polypeptide for the detection of a target ligand in a cell comprising a ligand-binding domain (LBD) capable of binding the target ligand, wherein the recombinant polypeptide has a longer half-life in the presence of the target ligand than in the absence of the target ligand.

2. The recombinant polypeptide of claim 1, further comprising a reporter molecule operably linked to the LBD.

3. The recombinant polypeptide of claim 2, wherein the reporter molecule is a screenable or selectable marker.

4. The recombinant polypeptide of claim 3, wherein the reporter molecule is a fluorescent molecule, luciferase, or an enzymatic component.

5. The recombinant polypeptide of claim 1, further comprising a DNA-binding domain (DBD) and a transcription activation domain (TAD), each in operable linkage with the LBD.

6. The recombinant polypeptide of claim 1, wherein the LBD is a naturally occurring polypeptide, or a variant or fragment thereof with ligand binding activity.

7. The recombinant polypeptide of claim 1, wherein the LBD is computationally designed to bind the target ligand.

8. The recombinant polypeptide of claim 7, wherein the LBD is computationally designed to include destabilizing mutations at a homodimer interface of a homodimeric protein.

9. The recombinant polypeptide of claim 7, wherein the LBD is computationally designed to include mutations that maintain protein structure while altering the specificity of ligand binding.

10. The recombinant polypeptide of claim 1, wherein the LBD comprises:
a) a polypeptide sequence comprising one or more mutations compared with DIG10.3 (SEQ ID NO:3) and having ligand binding activity; or
b) a fragment of DIG10.3 (SEQ ID NO:3) having ligand binding activity.

11. The recombinant polypeptide of claim 10, wherein the LBD comprises a polypeptide sequence comprising 1, 2, or 3 mutations compared with DIG10.3 (SEQ ID NO:3) and having ligand binding activity.

12. The recombinant polypeptide of claim 10, wherein the LBD is DIG₀ (SEQ ID NO:5), DIG₁ (SEQ ID NO:7), DIG₂ (SEQ ID NO:9), DIG₃ (SEQ ID NO:11), PRO₀ (SEQ ID NO:13), PRO₁ (SEQ ID NO:15), PRO₂ (SEQ ID NO:17), or PRO₃ (SEQ ID NO:19).

13. The recombinant polypeptide of claim 5, further comprising a degron.

14. The recombinant polypeptide of claim 13, wherein the degron is MATα.

15. The recombinant polypeptide of claim 5, wherein the DBD recognizes a naturally occurring or synthetic upstream activating sequence (UAS) within a promoter.

16. The recombinant polypeptide of claim 5, wherein the DBD comprises:
a) a polypeptide sequence of Gal4 (SEQ ID NO:25) or LexA (SEQ ID NO:29);
b) a polypeptide sequence comprising one or more mutations compared with Gal4 Gal4 (SEQ ID NO:25) or LexA (SEQ ID NO:29) and having DNA binding activity; or
c) a fragment of Gal4 (SEQ ID NO:25) or LexA (SEQ ID NO:29) having DNA binding activity.

17. The recombinant polypeptide of claim 16, wherein the DBD comprises the sequence of Gal4 (SEQ ID NO:25) or LexA (SEQ ID NO:29).

18. The recombinant polypeptide of claim 5, wherein the TAD comprises:
a) a polypeptide sequence of VP16 (SEQ ID NO:30) or VP64 (SEQ ID NO:31);
b) a polypeptide sequence comprising one or more mutations compared with VP16 (SEQ ID NO:30) or VP64 (SEQ ID NO:31) and having transcription activation activity; or
c) a fragment of VP16 (SEQ ID NO:30) or VP64 (SEQ ID NO:31) having transcription activation activity.

19. The recombinant polypeptide of claim 18, where the TAD comprises the sequence of VP16 (SEQ ID NO:30) or VP64 (SEQ ID NO:31).

20. The recombinant polypeptide of claim 5, wherein the DBD is G_{L77F} (SEQ ID NO:27), the LBD is PRO₁ (SEQ ID NO:15), and the TAD is VP16 (SEQ ID NO:30).

21. The recombinant polypeptide of claim 5, wherein the LBD comprises:
a) a polypeptide sequence of Fen49 (SEQ ID NO:23) or Fen 21 (SEQ ID NO:21);
b) a polypeptide sequence comprising one or more mutations compared with Fen49 (SEQ ID NO:23) or Fen 21 (SEQ ID NO:21) and having ligand binding activity; or
c) a fragment of Fen49 (SEQ ID NO:23) or Fen 21 (SEQ ID NO:21) having ligand binding activity.

22. The recombinant polypeptide of claim 21, wherein the LBD comprises a polypeptide sequence of Fen49 (SEQ ID NO:23) or Fen 21 (SEQ ID NO:21).

23. The recombinant polypeptide of claim 22, wherein the DBD is Gal4 (SEQ ID NO:25), the LBD is Fen49 (SEQ ID NO:23), and the TAD is VP16 (SEQ ID NO:30).

24. The recombinant polypeptide of claim 1, further comprising a Cas9 polypeptide sequence operably linked to the LBD.

25. A cell comprising the recombinant polypeptide of claim 1.

26. The cell of claim 25, wherein the cell is a mammalian, yeast, or plant cell.

27. A polynucleotide sequence encoding the polypeptide of claim 1.

28. A method of detecting the presence of a target molecule within a cell comprising introducing the recombinant polynucleotide of claim 1 into the cell and detecting the reporter molecule.


SEQUENCE LISTING (31 sequences)

SEQ ID NO: 1 - DNA, 8 bp, Artificial sequence
  Polynucleotide
ggsggsgg

SEQ ID NO: 2 - DNA, 435 bp, Artificial sequence
  Polynucleotide
atgaatgctaaagaaattgttgtccactcactgcgtctgctggaaaatggcgatgcccgtggttggtccgacctgtttcacccggaaggcgtgctggaatatccgtacgccccgccgggccataaaacccgttttgaaggtcgcgaaacgatttgggcgcacatgcgtctgttcccggaatatgtgaccgttcgctttacggatgtccagttctacgaaaccgccgatccggacctggcaatcggcgaatttcatggtgacggtgtcctcaccgcgagcggcggtaaactggcgtacgattatattgctgtttggcgtacgcgcgacggtcagatcctgctgtaccgtgtgtttttcaacccgctgcgtgtcctggaagctctgggcggtgtggaagcagctgcgaaaattgttcaaggcgcgggtagtctcgag

SEQ ID NO: 3 - protein, 145 aa, Artificial sequence
  Polypeptide
MNAKEIVVHSLRLLENGDARGWSDLFHPEGVLEYPYAPPGHKTRFEGRETIWAHMRLFPEYVTVRFTDVQFYETADPDLAIGEFHGDGVLTASGGKLAYDYIAVWRTRDGQILLYRVFFNPLRVLEALGGVEAAAKIVQGAGSLE

SEQ ID NO: 4 - DNA, 435 bp, Artificial sequence; identical to SEQ ID NO: 2
  Polynucleotide

SEQ ID NO: 5 - protein, 145 aa, Artificial sequence; identical to SEQ ID NO: 3
  Polypeptide

SEQ ID NO: 6 - DNA, 435 bp, Artificial sequence
  Polynucleotide
atgaatgctaaagaaattgttgtccactcactgcgtctgctggaaaatggcgatgcccgtggttggtccgacctgtttcacccggaaggcgtgctggaatatccgtacgccccgccgggccataaaacccgttttgaaggtcgcgaaacgatttgggcgcacatgcgtctgttcccggaatatgtgaccgttcgctttacggatgtccagttctacgaaaccgccgatccggacctggcaatcggcgtatttcatggtgacggtgtcctcaccgcgagcggcggtaaactggcgtacgattatattgctgtttggcgtacgcgcgacggtcagatcctgctgtaccgtgtgtttttcaacccgctgcgtgtcctggaagctctgggcggtgtggaagcagctgcgaaaattgttcaaggcgcgggtagtctcgag

SEQ ID NO: 7 - protein, 145 aa, Artificial sequence
  Polypeptide
MNAKEIVVHSLRLLENGDARGWSDLFHPEGVLEYPYAPPGHKTRFEGRETIWAHMRLFPEYVTVRFTDVQFYETADPDLAIGVFHGDGVLTASGGKLAYDYIAVWRTRDGQILLYRVFFNPLRVLEALGGVEAAAKIVQGAGSLE

SEQ ID NO: 8 - DNA, 435 bp, Artificial sequence
  Polynucleotide
atgaatgctaaagaaattgttgtccactcactgcgtctgctggaaaatggcgatgcccgtggttggtccgacctgtttcacccggaaggcgtgctggaatatccgcacgccccgccgggccataaaacccgttttgaaggtcgcgaaacgatttgggcgcacatgcgtctgttcccggaatatgtgaccgttcgctttacggatgtccagttctacgaaaccgccgatccggacctggcaatcggcgaatttcatggtgacggtgtcctcaccgcgagcggcggtaaactggcgtacgattatattgctgtttggcgtacgcgcgacggtcagatcctgctgtaccgtgtgtttttcaacccgctgcgtgtcctggaagctctgggcggtgtggaagcagctgcgaaaattgttcaaggcgcgggtagtctcgag

SEQ ID NO: 9 - protein, 145 aa, Artificial sequence
  Polypeptide
MNAKEIVVHSLRLLENGDARGWSDLFHPEGVLEYPHAPPGHKTRFEGRETIWAHMRLFPEYVTVRFTDVQFYETADPDLAIGEFHGDGVLTASGGKLAYDYIAVWRTRDGQILLYRVFFNPLRVLEALGGVEAAAKIVQGAGSLE

SEQ ID NO: 10 - DNA, 435 bp, Artificial sequence
  Polynucleotide
atgaatgctaaagaaattgttgtccactcactgcgtctgctggaaaatggcgatgcccgtggttggtccgacctgtttcacccggaaggcgtgctggaatatccgtacgccccgccgggccataaaacccgttttgaaggtcgcgaaacgatttgggcgcacatgcgtctgttcccggaatatgtgaccgttcgctttacggatgtccagttctacgaaaccgccgatccggacctggcaatcggcgaatttcatggtgacggtgtcctcaccgcgagcggcggtaaactggcgtacgattatattgctgtttggcgtacgcgcgacggtcagatcctgctgtaccgtgtgtttttcggcccgctgcgtgtcctggaagctctgggcggtgtggaagcagctgcgaaaattgttcaaggcgcgggtagtctcgag

SEQ ID NO: 11 - protein, 145 aa, Artificial sequence
  Polypeptide
MNAKEIVVHSLRLLENGDARGWSDLFHPEGVLEYPYAPPGHKTRFEGRETIWAHMRLFPEYVTVRFTDVQFYETADPDLAIGEFHGDGVLTASGGKLAYDYIAVWRTRDGQILLYRVFFGPLRVLEALGGVEAAAKIVQGAGSLE

SEQ ID NO: 12 - DNA, 435 bp, Artificial sequence
  Polynucleotide
atgaatgctaaagaaattgttgtccactcactgcgtctgctggaaaatggcgatgcccgtggttggtccgacctgtttcacccggaaggcgtgctggaatttccgtacgccccgccgggccataaaacccgttttgaaggtcgcgaaacgatttgggcgcacatgcgtctgttcccggaatatgtgaccgttcgctttacggatgtccagttctacgaaaccgccgatccggacctggcaatcggcgaatttcatggtgacggtgtcctcaccgcgagcggcggtaaactggcgttcgattttattgctgtttggcgtacgcgcgacggtcagatcctgctgtaccgtgtgtttttcaacccgctgcgtgtcctggaagctctgggcggtgtggaagcagctgcgaaaattgttcaaggcgcgggtagtctcgag

SEQ ID NO: 13 - protein, 145 aa, Artificial sequence
  Polypeptide
MNAKEIVVHSLRLLENGDARGWSDLFHPEGVLEFPYAPPGHKTRFEGRETIWAHMRLFPEYVTVRFTDVQFYETADPDLAIGEFHGDGVLTASGGKLAFDFIAVWRTRDGQILLYRVFFNPLRVLEALGGVEAAAKIVQGAGSLE

SEQ ID NO: 14 - DNA, 435 bp, Artificial sequence
  Polynucleotide
atgaatgctaaagaaattgttgtccgctcactgcgtctgctgggaaatggcgatgcccgtggttggtccgacctgtttcacccggaaggcgtgctggaatttccgtacgccccgccgggccataaaacccgttttgaaggtcgcgaaacgatttgggcgcacatgcgtctgttcccggaatatgtgacctttcgctttacggatgtccagttctacgaaaccgccgatccggacctggcaatcggcgaatttcatggtgacggtgtcctcaccacgagcggcggtaaactggcgttcgattttattgctgtttggcgtacgcgcgacggtcagatcctgctgtaccgtgtgtttttcaacccgctgcgtgtcctggaagctctgggcggtgtggaagcagctgcgaaaattgttcaaggcgcgggtagtctcgag

SEQ ID NO: 15 - protein, 145 aa, Artificial sequence
  Polypeptide
MNAKEIVVRSLRLLGNGDARGWSDLFHPEGVLEFPYAPPGHKTRFEGRETIWAHMRLFPEYVTFRFTDVQFYETADPDLAIGEFHGDGVLTTSGGKLAFDFIAVWRTRDGQILLYRVFFNPLRVLEALGGVEAAAKIVQGAGSLE

SEQ ID NO: 16 - DNA, 435 bp, Artificial sequence
  Polynucleotide
atgaatgctaaagaaattgttgtccactcactgcgtctgctggaaaatggcgatgcccgtggttggtccgacctgtttcacccggaaggcgtgctggaatttccgtacgccccgccgggccataaaacccgttttgaaggtcgcgaaacgatttgggcgcacatgcgtctgttcccggaatatgtgaccgttcgctttacggatgtccagttctacgaaaccgccgatccggacctggcaatcggcgaatttcatggtgacggtgtcctcaccgcgagcggcggtatgctggcgttcgattttattgctgtttggcgtacgcgcgacggtcagatcctgcagtaccgtgtgttttacaacccgctgcgtgaactggaagctctgggcggtgtggaagcagctgcgaaaattgttcaaggcgcgggtagtctcgag

SEQ ID NO: 17 - protein, 145 aa, Artificial sequence
  Polypeptide
MNAKEIVVHSLRLLENGDARGWSDLFHPEGVLEFPYAPPGHKTRFEGRETIWAHMRLFPEYVTVRFTDVQFYETADPDLAIGEFHGDGVLTASGGMLAFDFIAVWRTRDGQILQYRVFYNPLRELEALGGVEAAAKIVQGAGSLE

SEQ ID NO: 18 - DNA, 435 bp, Artificial sequence
  Polynucleotide
atgaatgctaaagaaattgttgtccactcactgcgtctgctggaaaatggcgatgcccgtggttggtccgacctgtttcacccggaaggcgtgctggaatttccgtacgccccgccgggccataaaacccgttttgaaggtcgcgaaacgatttgggcgcacatgcgtctgttcccggaatatgtgaccgttcgctttacggatgtccagttctacgaaaccgccgatccggacctggcaatcggcgaatttcatggtgacggtgtcctcaccgcgagcggcggtgaactggcgttcgattttattgctgcttggcgtacgcgcgacggtcagatcctgctgtaccgtgtgtttttcaacccgctgcgtgtcctggaagctctgggcggtgtggaagcagctgcgaaaattgttcaaggcgcgggtagtctcgag

SEQ ID NO: 19 - protein, 145 aa, Artificial sequence
  Polypeptide
MNAKEIVVHSLRLLENGDARGWSDLFHPEGVLEFPYAPPGHKTRFEGRETIWAHMRLFPEYVTVRFTDVQFYETADPDLAIGEFHGDGVLTASGGELAFDFIAAWRTRDGQILLYRVFFNPLRVLEALGGVEAAAKIVQGAGSLE

SEQ ID NO: 20 - DNA, 423 bp, Artificial sequence
  Polynucleotide
atgtccgaacaaatcgccgccgttagaagaatggtagaagcctataatactggtaaaaccgacgacgttgccgactacatccaccctgaatatatgtctccatacactttggaattcacttcattaagaggtcctgaattgttcgctatcgcagttgcctggttgaagaaatgggcttccgaagaagcaagagttgaagaagtaggtattgaagaaagagccgattgggttagagctagattggtcttatatggtagacacgtcggtgaaggtgttggtatggcaccaacaggtagattattttctggtgaacaaatccacttgttgcatttcgtagatggtaaaatccatcaccatagaatgtggcctgactacaccggtataaagagacaattgggtgaaccatggcctgaaactgaacat

SEQ ID NO: 21 - protein, 141 aa, Artificial sequence
  Polypeptide
MSEQIAAVRRMVEAYNTGKTDDVADYIHPEYMSPYTLEFTSLRGPELFAIAVAWLKKWASEEARVEEVGIEERADWVRARLVLYGRHVGEGVGMAPTGRLFSGEQIHLLHFVDGKIHHHRMWPDYTGIKRQLGEPWPETEH

SEQ ID NO: 22 - DNA, 555 bp, Artificial sequence
  Polynucleotide
atgtctaccgactactggctgaacttcaccgacggtggtggtatcgttaacgcggttaacggttctggtggtaactactctgttaactggtccaacaccggttctttcgttgttggtaaaggttggaccaccggttctccgttccgtaccatcaactacaacgcgggtgtttgggcgccgaacggttggggtgcgctggcgctggttggttggacccgttctccgctgatcgcgtactacgttgttgactcttggggtacctaccgttggaccggtacctacaaaggtaccgttaaatctgatggtggtacctacgacatctacaccaccacccgttacaacgcgccgtctatcgacggtgaccgtaccaccttcacccagtactggtctgttcgtcagtctaaacgtccgaccggttctaacgctaccatcaccttctctaaccacgttaacgcgtggaaatctcacggtatgaacctgggttctaactgggcgtaccaggttatggcgaccgcgggttaccagtcttctggttcttccaatgtgaccgtttgg

SEQ ID NO: 23 - protein, 185 aa, Artificial sequence
  Polypeptide
MSTDYWLNFTDGGGIVNAVNGSGGNYSVNWSNTGSFVVGKGWTTGSPFRTINYNAGVWAPNGWGALALVGWTRSPLIAYYVVDSWGTYRWTGTYKGTVKSDGGTYDIYTTTRYNAPSIDGDRTTFTQYWSVRQSKRPTGSNATITFSNHVNAWKSHGMNLGSNWAYQVMATAGYQSSGSSNVTVW

SEQ ID NO: 24 - DNA, 279 bp, Artificial sequence
  Polynucleotide
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgtta

SEQ ID NO: 25 - protein, 93 aa, Artificial sequence
  Polypeptide
MKLLSSIEQACDICRLKKLKCSKEKPKCAKCLKNNWECRYSPKTKRSPLTRAHLTEVESRLERLEQLFLLIFPREDLDMILKMDSLQDIKALL

SEQ ID NO: 26 - DNA, 279 bp, Artificial sequence
  Polynucleotide
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagactttgacatgattttgaaaatggattctttacaggatataaaagcattgtta

SEQ ID NO: 27 - protein, 93 aa, Artificial sequence
  Polypeptide
MKLLSSIEQACDICRLKKLKCSKEKPKCAKCLKNNWECRYSPKTKRSPLTRAHLTEVESRLERLEQLFLLIFPREDFDMILKMDSLQDIKALL

SEQ ID NO: 28 - DNA, 252 bp, Artificial sequence
  Polynucleotide
atgaaagcgttaacggccaggcaacaagaggtgtttgatctcatccgtgatcacatcagccagacaggtatgccgccgacgcgtgcggaaatcgcgcagcgtttggggttccgttccccaaacgcggctgaagaacatctgaaggcgctggcacgcaaaggcgttattgaaattgtttccggcgcatcacgcgggattcgtttattgcaggaagaggaagaagggttgccgctggtaggtcgtgtggctgcc

SEQ ID NO: 29 - protein, 84 aa, Artificial sequence
  Polypeptide
MKALTARQQEVFDLIRDHISQTGMPPTRAEIAQRLGFRSPNAAEEHLKALARKGVIEIVSGASRGIRLLQEEEEGLPLVGRVAA

SEQ ID NO: 30 - protein, 128 aa, Artificial sequence
  Polypeptide
AYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 31 - protein, 50 aa, Artificial sequence
  Polypeptide
DALDDFDLDMLGSDALDDFDLDMLGSDALDDFDLDMLGSDALDDFDLDML